(19) United States
(12) Patent Application Publication
(10) Pub. No.: US 2026/0265299 A1
(43) Pub. Date: Sep. 10, 2026

(54) SYNTHESIS OF BICYCLE TOXIN CONJUGATES, AND INTERMEDIATES THEREOF

(71) Applicant: BicycleTx Limited, Cambridge (GB)

(72) Inventors: Darren Limb, Cambridge (GB); William Moss, Cambridge (GB); David Witty, Cambridge (GB); Jie Zhang, Jiangsu (CN); Jinguang Lin, Jiangsu (CN); William Nesbit, Cambridge (GB)

(73) Assignee: BicycleTx Limited, Cambridge (GB)

(21) Appl. No.: 19/163,358

(22) PCT Filed: Mar. 8, 2024
(86) PCT No.: PCT/GB2024/050630

§ 371 Date: Sep. 8, 2025

(30) Foreign Application Priority Data
Mar. 9, 2023 (WO) PCT/CN2023/080496

(51) Int. Cl. C07K 5/103; A61K 47/64

(52) U.S. Cl. CPC C07K 5/1013; A61K 47/64

(57) ABSTRACT

The present invention relates methods of preparing Bicycle toxin conjugates, and intermediates thereof.

### FIELD OF THE INVENTION

The present invention relates to methods of synthesizing gvcMMAE, and methods of synthesizing Bicycle toxin conjugates (BTCs), for example, BT5528 and BT8009, comprising gvcMMAE (Glutaryl-Val-Cit-PAB-MMAE).

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å2; Wu et al. (2007), Science 330, 1066-71), a cyclic peptide with the Arg-Gly-Asp motif binding to integrin αVb3 (355 Å2) (Xiong et al. (2002), Science 296 (5565), 151-5) or the cyclic peptide inhibitor upain-1 binding to urokinase-type plasminogen activator (603 Å2; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher potential binding affinity. The reduced flexibility also leads to locking target-specific conformations, increasing binding specificity compared to linear peptides. This effect has been exemplified by a potent and selective inhibitor of matrix metalloproteinase 8, MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO2009/098450). Briefly, combinatorial libraries of linear peptides containing three cysteine residues and two regions of six random amino acids (Cys-(Xaa)6-Cys-(Xaa)6-Cys) were displayed on phage and cyclised by covalently linking the cysteine side chains to a small molecule (tris-(bromomethyl)benzene).

### SUMMARY OF THE INVENTION

The present invention provides methods of synthesizing Glutaryl-Val-Cit-PAB-MMAE (gvcMMAE).

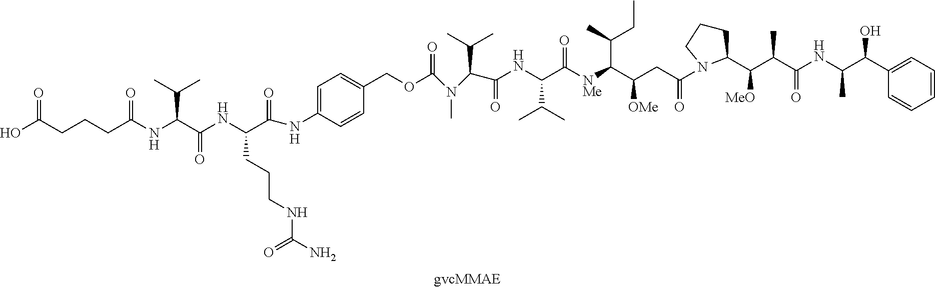

The present invention also provides methods of synthesizing a Bicycle toxin conjugate (BTC) comprising gvcMMAE. In some embodiments, a Bicycle toxin conjugate (BTC) is BT5528, or a pharmaceutically acceptable salt thereof. In some embodiments, a Bicycle toxin conjugate (BTC) is BT8009, or a pharmaceutically acceptable salt thereof.

### DETAILED DESCRIPTION OF THE INVENTION

### 1. General Description of Certain Aspects of the Invention

It has now been found that the method of synthesizing Glutaryl-Val-Cit-PAB-MMAE (gvcMMAE) from Val-Cit-PAB-MMAE (vcMMAE), as shown in Scheme I herein, can be improved for large scale production, including, for example, GMP production. For example, it has now been found that the method can be optimized by the following:
the equivalent of glutaric anhydride can be decreased from 1.2 eq. to about 1-1.1 eq. (e.g. about 1.1 eq.);
5.2 eq. of DIEA can be changed to about 1.3-1.5 eq. of TEA (e.g. about 1.3 eq. of TEA); and
DMA/THF mix solvent can be used as the reaction solvent.

In some embodiments the reaction temperature can be from about 15° C. to about 25° C. In some embodiments the reaction temperature can be decreased to 0° C.

The optimized method has been found to provide a more stable reaction condition. And the resulting reaction solution can be directly charged into anti-solvents (e.g. THF:MTBE; e.g. about 20:39 to about 25:30 v/v THF:MTBE, e.g. =about 1:2, e.g. about 75 v) to provide a good solid state of the product (gvcMMAE). This optimized process is found to be more suitable for scale up. Still further, it has been found that the disclosed method yields a gvcMMAE product having an improved purity profile.

Accordingly, in one aspect, the present invention provides a method of synthesizing gvcMMAE, comprising reacting vcMMAE with glutaric anhydride at the conditions as shown in Scheme I or Scheme II herein.

In another aspect, the present invention provides a method of synthesizing a Bicycle toxin conjugate (BTC), the method comprising reacting gvcMMAE with a Bicycle.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of each of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

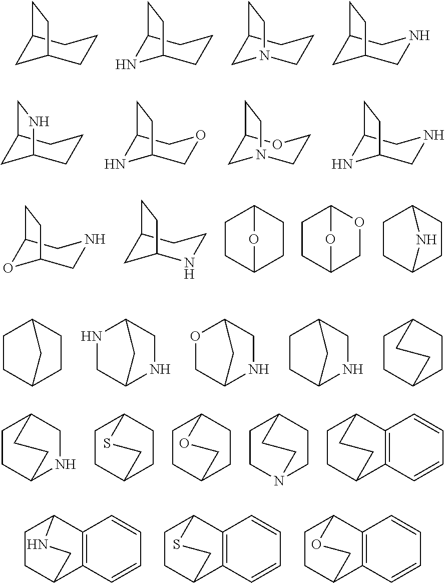

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group. A substituted alkynylene chain is a polymethylene group containing at least one triple bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

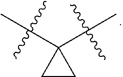 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘)}₂; —(CH₂)₀₋₄N(R^{∘})C(O)R^{∘}; —N(R^{∘})C(S)R^{∘}; —N(R^{∘})C(NR^{∘})N(R^{∘)}₂; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH) NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, (haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Additionally, pharmaceutically acceptable salts are described in detail in Pharmaceutical Salts: Properties, Selection, and Use, 2nd Revised Edition, (2011), P. Heinrich Stahl (Editor), Camille G. Wermuth (Editor), (ISBN: 978-3-906-39051-2), the entirety of which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, (C₁₋₆ alkyl) sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, condition, or disorder, to treat, diagnose, prevent, and/or delay the onset of the disease, condition, or disorder. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, condition, or disorder is the amount that alleviates, ameliorates, relieves, inhibits, prevents, delays onset of, reduces severity of and/or reduces incidence of one or more symptoms or features of the disease, condition, or disorder.

The terms “treat” or “treating,” as used herein, refers to partially or completely alleviating, inhibiting, delaying onset of, preventing, ameliorating and/or relieving a disease or disorder, or one or more symptoms of the disease or disorder. As used herein, the terms “treatment,” “treat,” and “treating” refer to partially or completely alleviating, inhibiting, delaying onset of, preventing, ameliorating and/or relieving a disease or disorder, or one or more symptoms of the disease or disorder, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

Bicycle toxin conjugate BT8009 has the structure shown below, and a preparation of BT8009 (BCY8245) is described in WO 2019/243832, the entirety of which is hereby incorporated herein by reference.

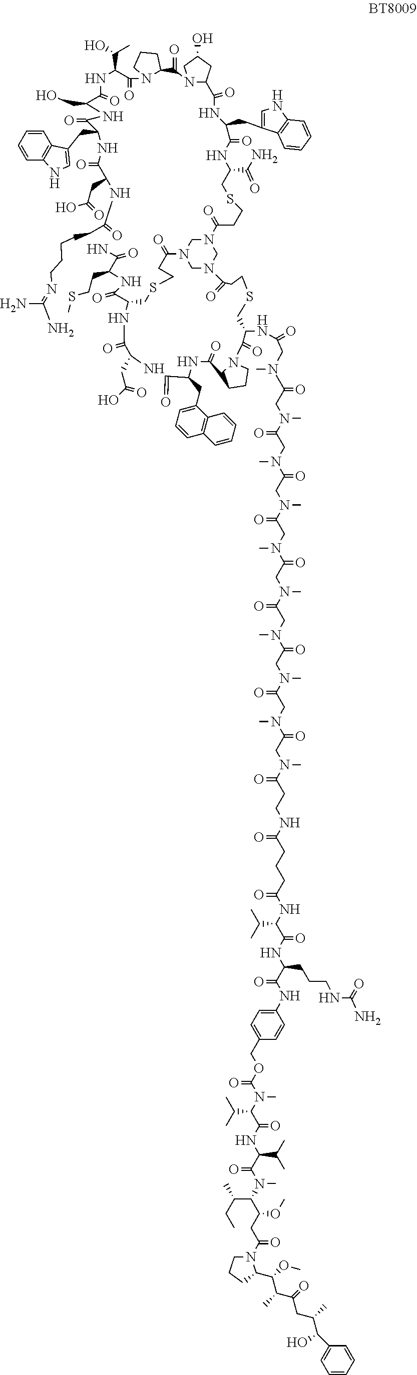

Bicycle toxin conjugate BT5528 has the structure shown below, wherein the molecular scaffold is 1,1′,1″-(1,3,5-triazinane-1,3,5-triyl)triprop-2-en-1-one (TATA), and the peptide ligand comprises the amino acid sequence (β-Ala)-Sar₁₀-A(HArg)D-Cᵢ(HyP)LVNPLCᵢᵢLHP(D-Asp)W(HArg)Cᵢᵢᵢ, wherein Sar is sarcosine, HArg is homoarginine, and HyP is hydroxyproline.

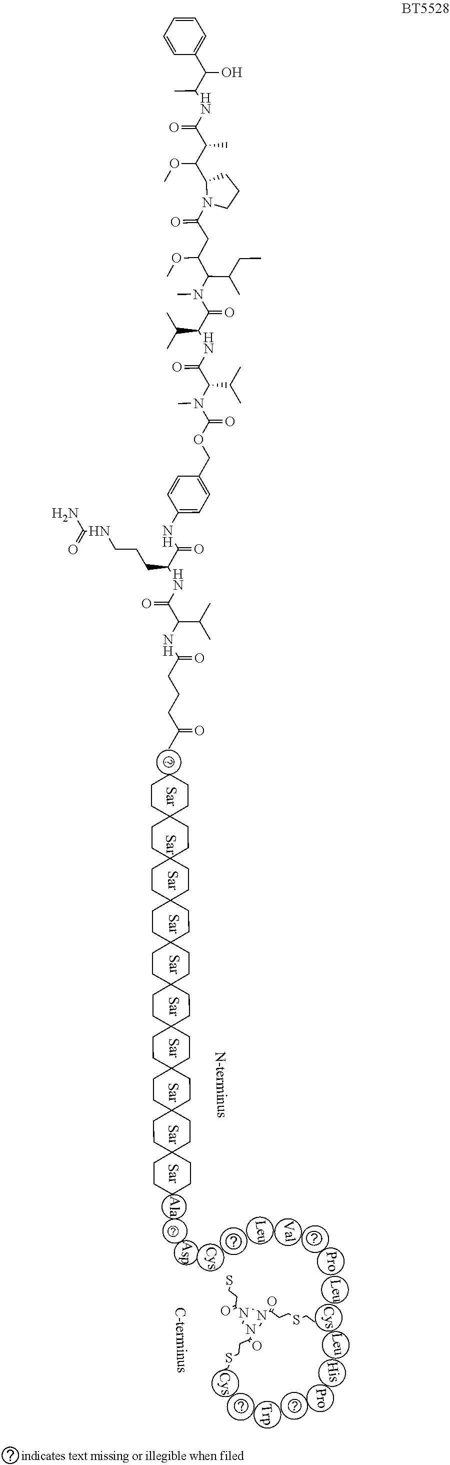

### 3. Description of Certain Embodiments of the Invention

In some embodiments, the present invention provides a method of synthesizing gvcMMAE, comprising reacting vcMMAE with glutaric anhydride. In some embodiments the present invention provides a method of synthesizing gvcMMAE, comprising reacting vcMMAE with glutaric anhydride in a solvent comprising N,N-dimethylacetamide (DMA) and tetrahydrofuran (THF). In some embodiments the present invention provides a method of synthesizing gvcMMAE, comprising reacting vcMMAE with glutaric anhydride in a solvent comprising N,N-dimethylacetamide (DMA) and tetrahydrofuran (THF) and optionally further comprising one or more further components, such as methyl tert-butyl ether (MTBE).

In some embodiments the method comprises adding glutaric anhydride into a solution comprising vcMMAE in a solvent comprising N,N-dimethylacetamide (DMA) and tetrahydrofuran (THF) to afford a reaction mixture. In some embodiments, the present invention provides a method of synthesizing gvcMMAE, comprising reacting vcMMAE with glutaric anhydride at the conditions as shown in Scheme I or Scheme II herein.

In some embodiments, the provided methods comprise reacting vcMMAE with from about 1 to about 1.1 equivalents of glutaric anhydride, relative to about 1 equivalent of vcMMAE. In some embodiments, glutaric anhydride is about 1.1 equivalent of vcMMAE. In some embodiments, glutaric anhydride is about 1 equivalent, about 1.01 equivalent, about 1.02 equivalent, about 1.03 equivalent, about 1.04 equivalent, about 1.05 equivalent, about 1.06 equivalent, about 1.07 equivalent, about 1.08 equivalent, or about 1.09 equivalent of vcMMAE. In some embodiments, glutaric anhydride is about 1.11 equivalent, about 1.12 equivalent, about 1.13 equivalent, about 1.14 equivalent, about 1.15 equivalent, about 1.16 equivalent, about 1.17 equivalent, about 1.18 equivalent, about 1.19 equivalent, or about 1.20 equivalent of vcMMAE. In some embodiments vcMMAE is reacted with from about 1 to about 1.20 equivalents of glutaric anhydride, such as from about 1 to about 1.1 equivalents of glutaric anhydride, e.g. from about 1.05 to about 1.15 equivalents of glutaric anhydride, e.g. from about 1.08 to about 1.12 equivalents of glutaric anhydride, such as about 1.1 equivalents of glutaric anhydride, relative to about 1 equivalent of vcMMAE.

In some embodiments, triethylamine (TEA) in the reaction mixture is about 1.3 equivalent of vcMMAE. In some embodiments, triethylamine (TEA) in the reaction mixture is about 1 equivalent to about 1.6 equivalent of vcMMAE, relative to about 1 equivalent of vcMMAE. In some embodiments the reaction mixture comprises from about 1.3 to about 1.5 equivalents of triethylamine. In some embodiments, triethylamine (TEA) in the reaction mixture is about 1 equivalent, about 1.05 equivalent, 1.10 equivalent, 1.15 equivalent, 1.2 equivalent, 1.25 equivalent, 1.3 equivalent, 1.35 equivalent, 1.4 equivalent, 1.45 equivalent, 1.5 equivalent, 1.55 equivalent, or 1.6 equivalent of vcMMAE. In some embodiments the reaction mixture comprises from about 1 to about 1.6 equivalents of TEA, such as from about 1.3 to about 1.5 equivalents of TEA, e.g. from about 1.3 to about 1.4 equivalents of TEA, e.g. about 1.3 equivalents of TEA, relative to about 1 equivalent of vcMMAE. In some embodiments the method comprises adding from about 1 to about 1.6 equivalents of TEA, such as from about 1.3 to about 1.5 equivalents of TEA, e.g. from about 1.3 to about 1.4 equivalents of TEA, e.g. about 1.3 equivalents of TEA to the reaction mixture.

In some embodiments, the reaction between vcMMAE and glutaric anhydride is in a solvent comprising N,N-dimethylacetamide (DMA) and tetrahydrofuran (THF). In some embodiments the solvent comprises DMA, THF and one or more further components. In some embodiments the solvent comprises DMA, THF and MTBE.

In some embodiments, the solvent comprises N,N-dimethylacetamide (DMA) and tetrahydrofuran (THF), or is a mixture of DMA and THE, at a volume ratio of about 1:15. In some embodiments the solvent comprises DMA and THE at a volume ratio of from about 1:10 to about 1:200, such as from about 1:50 to about 1:150, e.g. from about 1:100 to about 1:130, e.g. from about 1:110 to about 1:120, such as about 1:115. In some embodiments the solvent comprises DMA and THE at a volume ratio of from about 1:10 to about 1:20, such as from about 1:12 to about 1:18, e.g. from about 1:14 to about 1:16, e.g. about 1:15. In some embodiments, the solvent comprises N,N-dimethylacetamide (DMA) and tetrahydrofuran (THF), or is a mixture of DMA and THF, at a volume ratio of about 1:2, 1:3, 1:4, 1:5, 1:6, 1:7, 1:8, 1:9, 1:10, 1:11, 1:12, 1:13, 1:14, 1:16, 1:17, 1:18, 1:19, or 1:20.

In some embodiments the THF is present at about 3 to about 3.9 kg/kg. In some embodiments the THF is present at about 3 to about 3.9 kg/kg relative to the mass of vcMMAE. In some embodiments the DMA is present at about 0.2 to about 0.3 kg/kg. In some embodiments the DMA is present at about 0.2 to about 0.3 kg/kg relative to the mass of vcMMAE. In some embodiments the solvent comprises from about 0.2:3.9 to about 0.3-3 v/v DMA/THF, e.g. relative to the vcMMAE. In some embodiments, the solvent is a mixture of about 0.25:3.75 v/v DMA and THF (0.25:3.75 v/v DMA/THF). In some embodiments the solvent comprises from about 0.35:3.65 to about 0.15:3.85 v/v DMA/THF. In some embodiments the solvent comprises from about 0.3:3.7 to about 0.2:3.8 v/v DMA/THF. In some embodiments, the solvent is a mixture of about 0.20:3.80 v/v DMA and THF. In some embodiments, the solvent is a mixture of about 0.30:3.70 v/v DMA and THF. In some embodiments, the solvent is a mixture of about 0.15:3.85 v/v DMA and THF. In some embodiments, the solvent is a mixture of about 0.35:3.65 v/v DMA and THF.

In some embodiments the solvent comprises DMA, THF and MTBE. In some embodiments the solvent comprises DMA, THF and MTBE at a volume ratio of about 1:A:B DMA:THF:MTBE, wherein A is from about 10 to about 200, such as from about 50 to about 150, e.g. from about 100 to about 130, e.g. from about 110 to about 120, such as about 115; and B is from about 10 to about 500, e.g. from about 100 to about 300, e.g. from about 150 to about 250, e.g. from about 180 to about 220, e.g. about 200.

In some embodiments the solvent comprises DMA, THF and MTBE; the DMA is present at about 0.2 to about 0.3 v; the THF is present at about 25 to about 35 v; and the DMA is present at about 30 to about 80 v, e.g. relative to the vcMMAE. In some embodiments the DMA is present at about 0.23 to about 0.27 v (e.g. about 0.25 v); the THF is present at about 26 to about 30 v (e.g. about 28-29 v, e.g. about 28.75 v); and the DMA is present at about 40 to about 60 v/v (e.g. about 50 v), e.g. relative to the vcMMAE.

In some embodiments the ratio of the DMA:THF is from about 0.2:35 to about 0.3:25 v/v. In some embodiments the ratio of the DMA:THF is from about 0.23:30 to about 0.27:26 v/v (e.g. about 0.25:28-29 v/v, e.g. 0.25:28.75 v/v). In some embodiments the ratio of the DMA:MTBE is from about 0.2:80 to about 0.3:30 v/v. In some embodiments the ratio of the DMA:MTBE is from about 0.23:60 to about 0.27:40 v/v (e.g. about 0.25:50 v/v). In some embodiments the ratio of the THF:MTBE is from about 25:80 to about 35:30 v/v. In some embodiments the ratio of the THF:MTBE is from about 26:60 to about 30:40 v/v (e.g. about 28-29:50 v/v, e.g. 28.75:50 v/v).

In some embodiments, the reaction between vcMMAE and glutaric anhydride is in a solvent comprising dichloromethane (DCM). In some embodiments, the reaction between vcMMAE and glutaric anhydride is in a solvent, which is dichloromethane (DCM).

In some embodiments, the reaction between vcMMAE and glutaric anhydride is in a solvent comprising acetonitrile (MeCN). In some embodiments, the reaction between vcMMAE and glutaric anhydride is in a solvent, which is acetonitrile (MeCN).

In some embodiments, the reaction between vcMMAE and glutaric anhydride is in a solvent comprising 2-Methyltetrahydrofuran (2-MeTHF). In some embodiments, the reaction between vcMMAE and glutaric anhydride is in a solvent, which is 2-Methyltetrahydrofuran (2-MeTHF).

In some embodiments, the reaction between vcMMAE and glutaric anhydride is in a solvent comprising N,N-dimethylacetamide (DMA). In some embodiments, the reaction between vcMMAE and glutaric anhydride is in a solvent, which is N,N-dimethylacetamide (DMA).

In some embodiments, the reaction between vcMMAE and glutaric anhydride is in a solvent comprising N,N-dimethylacetamide (DMA) and acetonitrile (MeCN). In some embodiments, the solvent comprises N,N-dimethylacetamide (DMA) and acetonitrile (MeCN), or is a mixture of DMA and MeCN, at a volume ratio of about 1:3. In some embodiments, the solvent comprises N,N-dimethylacetamide (DMA) and acetonitrile (MeCN), or is a mixture of DMA and MeCN, at a volume ratio of about 1:1, 1:2, 1:4, 1:5, 1:6, 1:7, 1:8, 1:9, 1:10, 1:11, 1:12, 1:13, 1:14, or 1:15. In some embodiments, the solvent comprises N,N-dimethylacetamide (DMA) and acetonitrile (MeCN), or is a mixture of DMA and MeCN, at a volume ratio of about 10:1, 9:1, 8:1, 7:1, 6:1, 5:1, 4:1, 3:1, 2:1, or 1.5:1.

In some embodiments, the reaction between vcMMAE and glutaric anhydride is carried out at a temperature of from about-5 to about 25° C. In some embodiments the reaction is carried out at a temperature of from about 15° C. to about 25° C. In some embodiments the reaction is carried out at a temperature of from about 18° C. to about 22° C., such as about 20° C. In some embodiments the reaction between vcMMAE and glutaric anhydride is conducted over a period of from about 10 minutes to about 5 hours, e.g. about 10 minutes to about 2 hours, such as from about 30 minutes to about 1.5 hours, e.g. about for an hour. In some embodiments the reaction is conducted over a period of from about 1 to about 3 hours. In some embodiments the reaction is conducted until completion.

In some embodiments the reaction comprises stirring the reaction mixture. In some embodiments the reaction comprises stirring the reaction mixture at a temperature of from about 15° C. to about 25° C., e.g. from about 18° C. to about 22° C., such as about 20° C. for a period of from about 10 minutes to about 5 hours, 10 minutes to about 2 hours, such as from about 30 minutes to about 1.5 hours, e.g. about for an hour; or for about 1 to about 3 hours. In some embodiments the reaction mixture is stirred until the reaction has gone to completion. In some embodiments a stirring rate is about 10 rpm to about 1000 rpm.

In some embodiments, the reaction between vcMMAE and glutaric anhydride is carried out at about 0° C. In some embodiments, the reaction between vcMMAE and glutaric anhydride is carried out at about −5° C. to about 5° C. In some embodiments, the reaction between vcMMAE and glutaric anhydride is carried out at about −5° C., −4° C., −3° C., −2° C., or −1° C. In some embodiments, the reaction between vcMMAE and glutaric anhydride is carried out at about 1° C., 2° C., 3° C., 4° C., or 5° C.

In some embodiments, the present invention provides a method of synthesizing gvcMMAE, the method comprising reacting from about 1 to about 1.1 equivalent glutaric anhydride with about 1 equivalent vcMMAE in a solvent comprising from about 0.2:3.9 to about 0.3-3 v/v DMA/THF at a temperature of from about 15 to about 25° C.

In some embodiments, the present invention provides a method of synthesizing gvcMMAE, the method comprising adding about 1 to about 1.1 (e.g. about 1.1) equivalent glutaric anhydride to a solution comprising about 1 equivalent vcMMAE in a solvent comprising from about 0.2:3.9 v/v to about 0.3-3 v/v DMA/THF (e.g. about 0.25:3.75 v/v DMA/THF) and about 1.3 to about 1.5 equivalents of triethylamine, wherein the method comprises conducting the reaction at a temperature of from about 15 to about 25° C. (e.g. about 20° C.) for a period of from about 10 minutes to about 2 hours.

In some embodiments, the present invention provides a method of synthesizing gvcMMAE, the method comprising adding about 1 to about 1.1 (e.g. about 1.1) equivalent glutaric anhydride to a solution comprising about 1 equivalent vcMMAE in a solvent comprising from about 0.2:3.9 to about 0.3-3 v/v DMA/THF (e.g. about 0.25:3.75 v/v DMA/THF) to afford a reaction mixture; adding about 1.3 to about 1.5 equivalents of triethylamine; and stirring the reaction mixture for a period of from about 10 minutes to about 2 hours (e.g. from about 30 minutes to about 1.5 hours, e.g. about for an hour) at a temperature of from about 15 to about 25° C. (e.g. about 20° C.).

In some embodiments, the present invention provides a method of synthesizing gvcMMAE, the method comprising reacting from about 1 to about 1.1 equivalent glutaric anhydride with about 1 equivalent vcMMAE in a solvent comprising DMA, THF and MTBE at a volume ratio of about 1:A:B DMA:THF:MTBE, wherein A is from about 10 to about 200 and B is from about 10 to about 500; at a temperature of from about 15 to about 25° C.

In some embodiments, the present invention provides a method of synthesizing gvcMMAE, the method comprising adding about 1 to about 1.1 (e.g. about 1.1) equivalent glutaric anhydride to a solution comprising about 1 equivalent vcMMAE in a solvent comprising DMA, THF and MTBE, wherein the DMA is present at about 0.2 to about 0.3 v; the THF is present at about 25 to about 35 v; and the DMA is present at about 30 to about 80 v, e.g. relative to the vcMMAE, and about 1.3 to about 1.5 equivalents of triethylamine, wherein the method comprises conducting the reaction at a temperature of from about 15 to about 25° C. (e.g. about 20° C.) for a period of from about 10 minutes to about 5 hours.

In some embodiments, the present invention provides a method of synthesizing gvcMMAE, the method comprising adding about 1 to about 1.1 (e.g. about 1.1) equivalent glutaric anhydride to a solution comprising about 1 equivalent vcMMAE in a solvent comprising DMA, THF and MTBE, wherein the DMA is present at about 0.23 to about 0.27 v (e.g. about 0.25 v); the THF is present at about 26 to about 30 v (e.g. about 28-29 v, e.g. about 28.75 v); and the DMA is present at about 40 to about 60 v/v (e.g. about 50 v), e.g. relative to the vcMMAE, to afford a reaction mixture; adding about 1.3 to about 1.5 equivalents of triethylamine; and stirring the reaction mixture for a period of from about 1 to about 3 hours at a temperature of from about 15 to about 25° C. (e.g. about 20° C.).

In some embodiments, the present invention provides a method of synthesizing gvcMMAE, the method comprising adding about 1.1 equivalent glutaric anhydride into a solution comprising about 1 equivalent vcMMAE in a solvent of about 0.25:3.75 v/v DMA and THF (0.25:3.75 v/v DMA/THF) at about −5° C. to about 5° C.

In some embodiments, the method comprises comprising quenching the reaction between vcMMAE and glutaric anhydride with water (H₂O). In some embodiments quenching the reaction comprises adding from about 0.002 to about 0.01 kg/kg water to the reaction mixture. In some embodiments quenching the reaction comprises adding from about 0.002 to about 0.01 kg/kg water relative to the mass of vcMMAE. In some embodiments quenching the reaction comprises adding from about 0.004 to about 0.008 kg/kg water, such as from about 0.005 to about 0.007 kg/kg water.

In some embodiments quenching the reaction comprises adding from about 0.1 to about 1 equivalents of water to the reaction mixture. In some embodiments quenching the reaction comprises adding from about 0.1 to about 1 equivalents of water (relative to the mass of vcMMAE) to the reaction mixture. In some embodiments quenching the reaction comprises adding from about 0.2 to about 0.6 equivalents of water, such as from about 0.3 to about 0.5 equivalents of water to the reaction mixture.

In some embodiments the reaction between vcMMAE and glutaric anhydride is quenched with water at a temperature of from about 15 to about 25° C. In some embodiments the reaction is quenched at a temperature of from about 18° C. to about 22° C., such as about 20° C. In some embodiments the reaction is quenched for a period of from about 1 minute to about 2 hours, such as from about 2 minutes to about 1 hour, e.g. from about 5 minutes to about 30 minutes, e.g. about 8 minutes to about 20 minutes, such as about 10 minutes.

In some embodiments quenching the reaction comprises stirring the reaction mixture. In some embodiments quenching the reaction comprises stirring the reaction mixture at a temperature of from about 15 to about 25° C., such as from about 18° C. to about 22° C., such as about 20° C. In some embodiments quenching the reaction mixture comprises stirring the reaction mixture for a period of from about 1 minute to about 2 hours, such as from about 2 minutes to about 1 hour, e.g. from about 5 minutes to about 30 minutes, e.g. about 8 minutes to about 20 minutes, such as about 10 minutes. In some embodiments a stirring rate is about 10 rpm to about 1000 rpm.

In some embodiments, therefore, the present invention provides a method of synthesizing gvcMMAE, the method comprising reacting from about 1 to about 1.1 equivalent glutaric anhydride with about 1 equivalent vcMMAE in a solvent comprising from about 0.2:3.9 to about 0.3-3 v/v DMA/THF at a temperature of from about 15 to about 25° C.; and quenching the reaction with water.

In some embodiments, the present invention provides a method of synthesizing gvcMMAE, the method comprising adding about 1 to about 1.1 (e.g. about 1.1) equivalent glutaric anhydride to a solution comprising about 1 equivalent vcMMAE in a solvent comprising from about 0.2:3.9 v/v to about 0.3-3 v/v DMA/THF (e.g. about 0.25:3.75 v/v DMA/THF) and about 1.3 to about 1.5 equivalents of triethylamine, wherein the method comprises stirring the reaction mixture at a temperature of from about 15 to about 25° C. (e.g. about 20° C.); and quenching the reaction by adding from about 0.002 to about 0.01 kg/kg water or from about 0.1 to about 1 equivalents of water to the reaction mixture.

In some embodiments, the present invention provides a method of synthesizing gvcMMAE, the method comprising adding about 1 to about 1.1 (e.g. about 1.1) equivalent glutaric anhydride to a solution comprising about 1 equivalent vcMMAE in a solvent comprising from about 0.2:3.9 to about 0.3-3 v/v DMA/THF (e.g. about 0.25:3.75 v/v DMA/THF) to afford a reaction mixture; adding about 1.3 to about 1.5 equivalents of triethylamine; and stirring the reaction mixture for a period of from about 10 minutes to about 2 hours at a temperature of from about 15 to about 25° C. (e.g. about 20° C.); and quenching the reaction by adding from about 0.002 to about 0.01 kg/kg water or from about 0.1 to about 1 equivalents of water to the reaction mixture at a temperature of from about 15 to about 25° C.

In some embodiments, the present invention provides a method of synthesizing gvcMMAE, the method comprising adding about 1.1 equivalent glutaric anhydride into a solution comprising about 1 equivalent vcMMAE in a solvent of about 0.25:3.75 v/v DMA and THF (0.25:3.75 v/v DMA/THF) at about −5° C. to about 5° C. to afford a reaction mixture, followed by adding about 1.3 equivalent TEA into the reaction mixture. In some embodiment, the reaction mixture is stirred for about 1 hour at about −5° C. to about 5° C. for the reaction to complete (i.e., the reaction mixture becomes a mixture comprising primarily gvcMMAE, or a gvcMMAE mixture). As used herein, unless implied otherwise by the context the term “gvcMMAE mixture” typically relates to the product of the reaction of vcMMAE and glutaric anhydride and typically comprises gvcMMAE and optionally residual reaction components such as residual solvent e.g. DMA, THF and/or TEA, and further optionally may comprise any unreacted vcMMAE and/or glutaric anhydride.

In some embodiments, a gvcMMAE mixture is warmed up to about 15° C. to about 25° C. In some embodiments, a gvcMMAE mixture is warmed up to about 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C., after the reaction is complete. In some embodiments, a gvcMMAE mixture is warmed up until it is a clear solution.

In some embodiments, the gvcMMAE mixture is added into an antisolvent to afford gvcMMAE as a precipitate. In some embodiments, the gvcMMAE mixture is added into a mixture of THF and MTBE to afford gvcMMAE as a precipitate. In some embodiments the gvcMMAE mixture is at about 15° C. to about 25° C. In some embodiments the gvcMMAE mixture is a clear solution. In some embodiments, a gvcMMAE mixture, which is a clear solution and at about 15° C. to about 25° C., is added into a mixture of THF and MTBE at about −5° C. to about 5° C. to afford gvcMMAE as a precipitate.

In some embodiments the mixture of THF and MTBE comprises about 20 to about 25 kg/kg THF. In some embodiments the mixture of THF and MTBE comprises about 20 to about 25 kg/kg THF relative to the mass of vcMMAE. In some embodiments the mixture of THF and MTBE comprises about 35 to about 39 kg/kg MTBE. In some embodiments the mixture of THF and MTBE comprises about 35 to about 39 kg/kg MTBE relative to the mass of vcMMAE. In some embodiments the mixture of THF and MTBE comprises from about 20:39 to about 25:30 v/v THF/MTBE. In some embodiments, a mixture of THF and MTBE is about 1:2 v/v THF and MTBE (1:2 v/v THF/MTBE). In some embodiments, a mixture of THF and MTBE is about 0.5:2.5, 0.6:2.4, 0.7:2.3, 0.8:2.2, 0.9:2.1, 1.1:1.9, 1.2:1.8, 1.3:1.7, 1.4:1.6, or 1:1 v/v THF and MTBE. In some embodiments the mixture of THF and MTBE comprises from about 0.5:2.5 to about 1:1 v/v THF and MTBE.

In some embodiments, the gvcMMAE mixture is added into a mixture of THF and MTBE at about −5° C. to about 5° C. to afford gvcMMAE as a precipitate. In some embodiments the gvcMMAE mixture is added into a mixture of THF and MTBE comprising from about 20:39 to about 25:30 v/v THF/MTBE at about −5° C. to about 5° C. to afford gvcMMAE as a precipitate. In some embodiments the gvcMMAE mixture is added (e.g. dropwise) into a mixture of THF and MTBE comprising from about 20:39 to about 25:30 v/v THF/MTBE at about −5° C. to about 5° C. with stirring to afford gvcMMAE as a precipitate, wherein the volume ratio of the DMA/THF mixture to the THF/MTBE mixture is from about 4:20 to about 4:200, e.g. from about 4:40 to about 4:100, e.g. from about 4:50 to about 4:75, such as about 4:75.

In some embodiments, the gvcMMAE mixture is added slowly into a mixture of THF and MTBE at about-5° C. to about 5° C. to afford gvcMMAE as a precipitate. In some embodiments, the gvcMMAE mixture is added dropwise into a mixture of THF and MTBE at about −5° C. to about 5° C. with stirring to afford gvcMMAE as a precipitate. In some embodiments, the volume ratio of the mixture of DMA and THE to the mixture of THF and MTBE is from about 4:20 to about 4:200, e.g. from about 4:40 to about 4:100, e.g. from about 4:50 to about 4:75, such as about 4:75. In some embodiments, the volume ratio of the mixture of DMA and THF to the mixture of THF and MTBE is 4:75. In some embodiments, the volume ratio of the 0.25:3.75 v/v DMA/THF mixture to the 1:2 v/v THF/MTBE mixture is about 4:75. In some embodiments, the gvcMMAE mixture is added slowly into a mixture of about 1:2 v/v THF and MTBE at about −5° C. to about 5° C. to afford gvcMMAE as a precipitate. In some embodiments, the gvcMMAE mixture is added dropwise into a mixture of about 1:2 v/v THF and MTBE at about −5° C. to about 5° C. with stirring to afford gvcMMAE as a precipitate. In some embodiments, the volume ratio of the mixture of DMA and THF to the mixture of THF and MTBE is 4:75. In some embodiments, the volume ratio of the 0.25:3.75 v/v DMA/THE mixture to the 1:2 v/v THF/MTBE mixture is about 4:75.

In some embodiments the gvcMMAE mixture in the THF/MTBE mixture is stirred for about 10 minutes to about 2 hours, such as from about 30 minutes to about 1.5 hours, e.g. about for an hour. In some embodiments a stirring rate is about 10 rpm to about 1000 rpm.

In some embodiments, a gvcMMAE precipitate as described above is filtered out as a wet filter cake, which is rinsed with MTBE. In some embodiments the gvcMMAE precipitate is rinsed with from about 3 to about 10 kg/kg MTBE. In some embodiments the gvcMMAE precipitate is rinsed with from about 3 to about 5 kg/kg MTBE. In some embodiments, 10 volume MTBE (where the 0.25:3.75 v/v DMA/THE mixture is 4 volume) is used to rinse the wet filter cake gvcMMAE.

In some embodiments, the gvcMMAE precipitate is dried at about 35° C. to about 45° C. after rinsing with MTBE. In some embodiments, the gvcMMAE precipitate is dried at 40° C. after rinsing with MTBE. In some embodiments the gvcMMAE precipitate is dried for about 1 hour to about 72 hours, such as from about 5 hours to about 48 hours, e.g. for about 15 hours to about 32 hours, e.g. about 24 hours.

In some embodiments, therefore, a method according to the present invention comprises:
i) dissolving 1 eq vc-PAB-MMAE in from about 3-3.9 kg/kg THF and about 0.2-0.3 kg/kg DMA at 15-25° C.;
ii) adding about 1-1.1 eq. glutaric anhydride and about 1.3-1.5 eq. TEA at 15-25° C.;
iii) quenching the reaction by adding about 0.002-0.01 kg/kg water to the reaction at 15-25° C. to yield a product solution;
iv) transferring the product solution to a mixture of about 20-25 kg/kg THF and about 35-39 kg/kg MTBE at from about-5 to about +5° C.;
v) isolating the product by filtration;
vi) optionally washing the product with 3-5 kg/kg MTBE and/or drying the product, e.g. under vacuum.

In some embodiments, the method comprises:
i) dissolving 1 eq vc-PAB-MMAE in from about 3-3.9 kg/kg THF and about 0.2-0.3 kg/kg DMA at 15-25° C. with stirring;
ii) adding about 1-1.1 eq. (e.g. about 1.1 eq.) glutaric anhydride and about 1.3-1.5 eq. (e.g. about 1.3 eq.) TEA at 15-25° C.; and stirring the reaction mixture to reaction completion, e.g. for about 0.5 to about 1.5 hours;
iii) quenching the reaction by adding about 0.002-0.01 kg/kg water to the reaction at 15-25° C. with stirring to yield a product solution;
iv) transferring the product solution to a mixture of about 20-25 kg/kg THF and about 35-39 kg/kg MTBE dropwise at from about-5 to about +5° C.;
v) isolating the product by filtration;
vi) washing the product with about 3-5 kg/kg MTBE; and drying the product under vacuum at about 35 to about 45° C.

In some embodiments, a method according to the present invention comprises:
i) dissolving 1 eq. vc-PAB-MMAE in a solvent comprising DMA, THF and MTBE at a volume ratio of about 1:A:B DMA:THF:MTBE, wherein A is from about 100 to about 130 (e.g. about 115); and B is from about 100 to about 300 (e.g. about 200), at about 15-25° C.;
ii) adding about 1-1.1 eq. (e.g. about 1.1 eq.) glutaric anhydride and about 1.3-1.5 eq. (e.g. about 1.3 eq.) TEA at 15-25° C. to yield a product solution;
iii) precipitating gvcMMAE from the product solution;
iv) isolating the product by filtration; and
v) optionally washing the product with about 3-5 kg/kg MTBE; and/or drying the product, e.g. under vacuum.

In some embodiments, the method comprises:
i) dissolving 1 eq. vc-PAB-MMAE in a solvent comprising DMA, THF and MTBE wherein the DMA is present at about 0.2 to about 0.3 v (e.g. about 25 v); the THF is present at about 25 to about 35 v (e.g. about 28-29 v); and the DMA is present at about 30 to about 80 v (e.g. about 50 v), e.g. relative to the vcMMAE
ii) adding about 1-1.1 eq. (e.g. about 1.1 eq.) glutaric anhydride and about 1.3-1.5 eq. (e.g. about 1.3 eq.) TEA at 15-25° C.; and stirring the reaction mixture e.g. to reaction completion, e.g. for about 1 to about 3 hours to yield a product solution;
iii) precipitating gvcMMAE from the product solution (e.g. by transferring the product solution to a mixture of about 20-25 kg/kg THF and about 35-39 kg/kg MTBE dropwise at from about-5 to about +5° C.);
iv) isolating the product by filtration; and
v) washing the product with about 3-5 kg/kg MTBE; and drying the product under vacuum at about 35 to about 45° C.

In some embodiments, the present invention provides a compound gvcMMAE, or a salt thereof. In some embodiments, the present invention provides a compound gvcMMAE, obtained by or obtainable by a method as disclosed herein.

In some embodiments, the present invention provides a compound of Formula III:

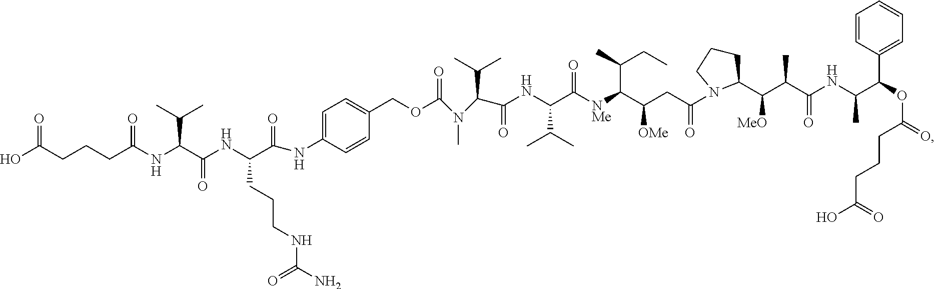

or a salt thereof.

In some embodiments, the present invention provides a composition comprising gvcMMAE, or a salt thereof, further comprising a compound of Formula III, or a salt thereof, as an impurity. In some embodiments, a composition comprising gvcMMAE, or a salt thereof, comprises less than about 1% of a compound of Formula III, or a salt thereof. In some embodiments, a composition comprising gvcMMAE, or a salt thereof, comprises less than about 0.9%, 0.8%, 0.7%, 0.6%, 0.5%, 0.4%, 0.3%, 0.2%, or 0.1% of a compound of Formula III, or a salt thereof.

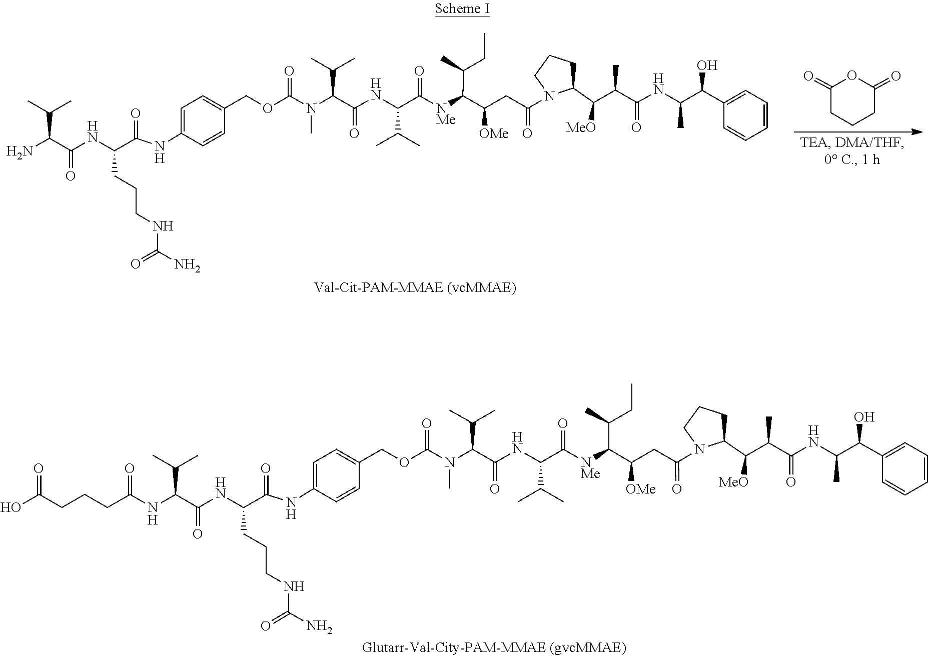 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

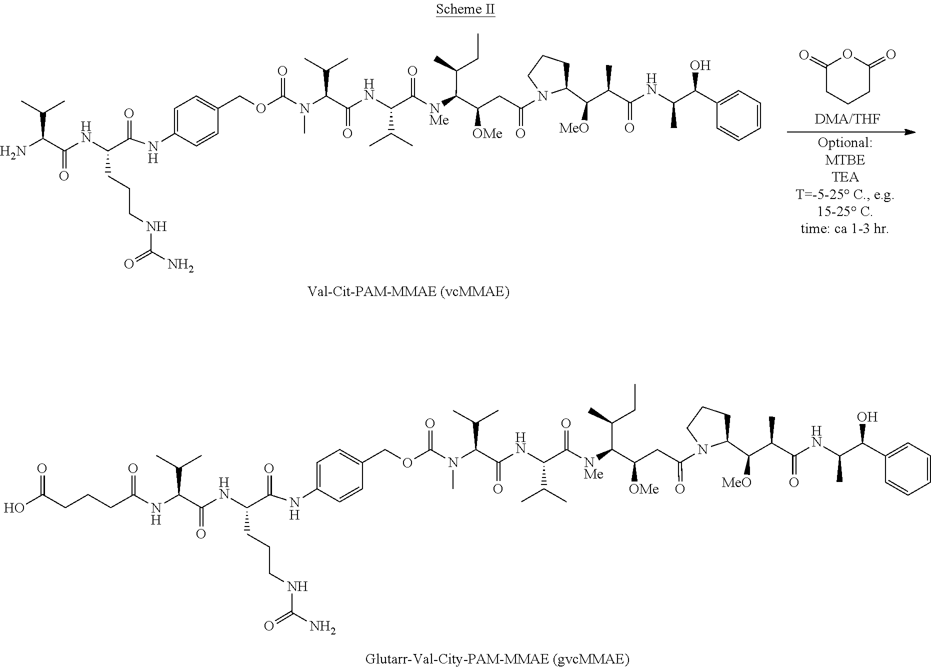 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

In some embodiments, the present invention provides a method of synthesizing a Bicycle toxin conjugate (BTC), the method comprising reacting gvcMMAE with a Bicycle.

In some embodiments, a Bicycle is a bicyclic peptide. In some embodiments, a Bicycle is a constrained bicyclic peptide that binds with high affinity and specificity to Nectin-4. In some embodiments, the bicyclic peptide is selected from those described in International Patent Application No. PCT/GB2019/051740 (International Publication No. WO 2019/243832), the entirety of which is incorporated herein by reference.

In some embodiments, a Bicycle is a constrained bicyclic peptide that binds with high affinity and specificity to Eph receptor tyrosine kinase A2 (EphA2). In some embodiments, the bicyclic peptide is selected from those described in International Patent Application Nos. PCT/GB2018/053675 (International Publication No. WO 2019/122860) and PCT/GB2018/053678 (International Publication No. WO 2019/122863), the entirety of each of which is incorporated herein by reference.

In some embodiments, the bicyclic peptide is:

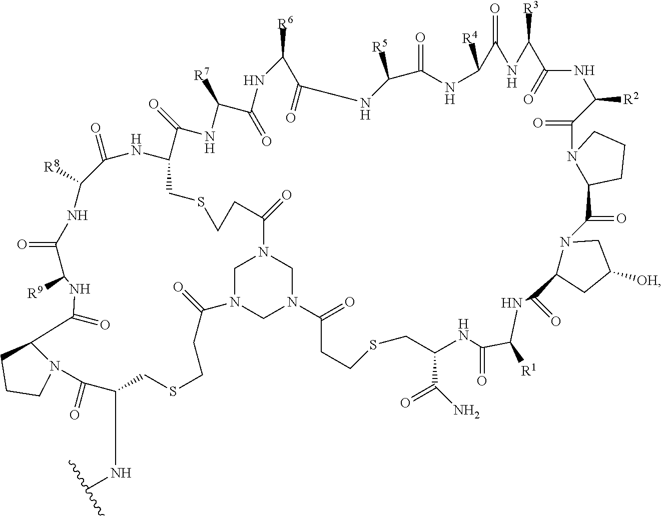

wherein each of R¹, R², R³, R⁴, R⁵, R⁶, R⁷, R⁸, and R⁹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In certain embodiments, R¹ is hydrogen or optionally substituted C₁₋₆ aliphatic. In certain embodiments, R¹ is

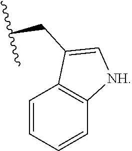

In certain embodiments, R² is hydrogen or optionally substituted C₁₋₆ aliphatic. In certain embodiments, R² is

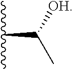

In certain embodiments, R³ is hydrogen or optionally substituted C₁₋₆ aliphatic. In certain embodiments, R³ is

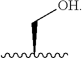

In certain embodiments, R⁴ is hydrogen or optionally substituted C₁₋₆ aliphatic. In certain embodiments, R⁴ is

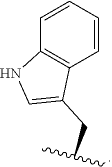

In certain embodiments, R⁵ is hydrogen or optionally substituted C₁₋₆ aliphatic. In certain embodiments, R⁵ is

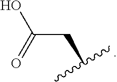

In certain embodiments, R⁶ is hydrogen or optionally substituted C₁₋₆ aliphatic. In certain embodiments, R⁶ is

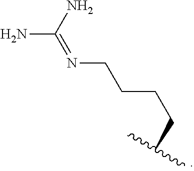

In certain embodiments, R⁷ is hydrogen or optionally substituted C₁₋₆ aliphatic. In certain embodiments, R⁷ is

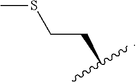

In certain embodiments, R⁸ is hydrogen or optionally substituted C₁₋₆ aliphatic. In certain embodiments, R⁸ is

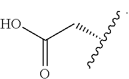

In certain embodiments, R⁹ is hydrogen or optionally substituted C₁₋₆ aliphatic. In certain embodiments, R⁹ is

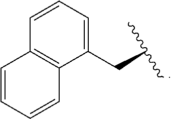

In some embodiments, a Bicyclic is of Formula II:

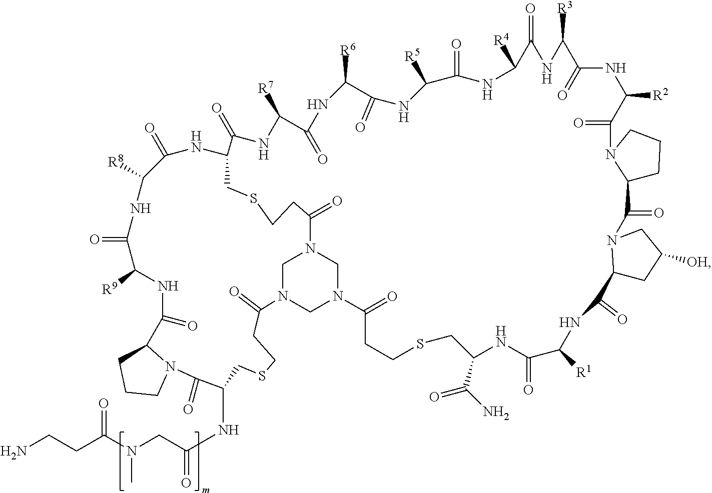

or a salt thereof, wherein each of R¹, R², R³, R⁴, R⁵, R⁶, R⁷, R⁸, and R⁹ is as defined below and described in embodiments herein, both singly and in combination, and m is 0, 1, 2, 3, 4, 5, 6, 7, 8, 9, 10, 11, 12, 13, 14, or 15.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15.

In some embodiments, a Bicycle toxin conjugate is of Formula I:

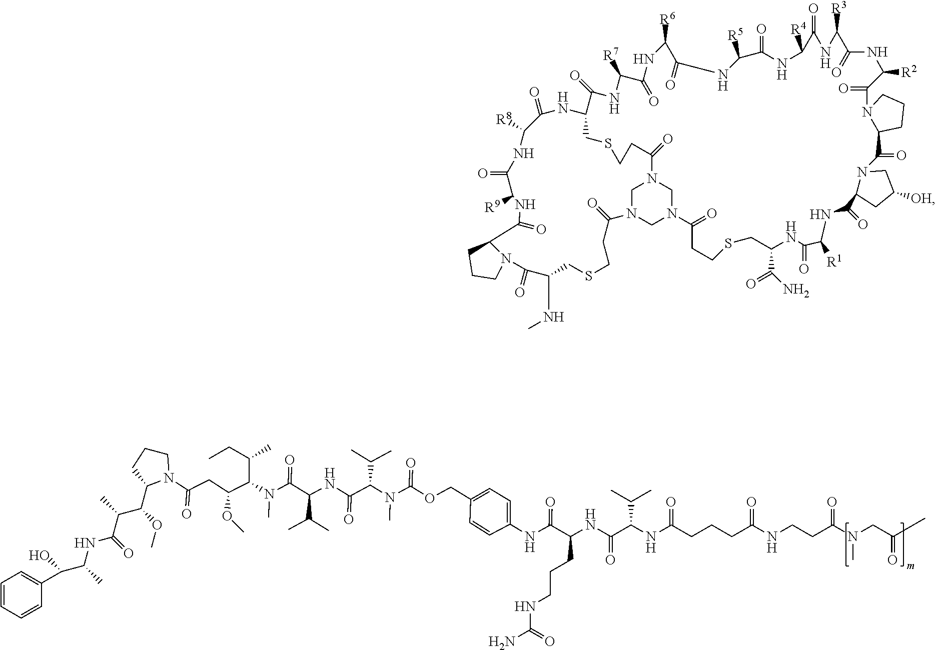

or a pharmaceutically acceptable salt thereof, wherein each of R¹, R², R³, R⁴, R⁵, R⁶, R⁷, R⁸, R⁹, and m is as defined below and described in embodiments herein, both singly and in combination.

In some embodiments, the present invention provides a method of synthesizing a Bicycle toxin conjugate (BTC) of Formula I, the method comprising reacting gvcMMAE with a Bicycle of Formula II.

In some embodiments, a Bicycle toxin conjugate of formula I is BT8009, or a pharmaceutically acceptable salt thereof.

In some embodiments, a Bicycle toxin conjugate of formula I is BT5528, or a pharmaceutically acceptable salt thereof.

In some embodiments, the present invention provides a Bicycle toxin conjugate (or a salt thereof) obtained by or obtainable by a method as disclosed herein. Also provided is a composition comprising a Bicycle toxin conjugate or a salt thereof and comprising less than 1% (e.g. less than about 0.9%, 0.8%, 0.7%, 0.6%, 0.5%, 0.4%, 0.3%, 0.2%, or 0.1%) of a compound of Formula III, or a salt thereof.

### EXEMPLIFICATION

The following Examples illustrate the invention described above; they are not, however, intended to limit the scope of the invention in any way. The beneficial effects of the pharmaceutical compounds, combinations, and compositions of the present invention can also be determined by other test models known as such to the person skilled in the pertinent art.

### List of Common Abbreviations Used in the Experimental Section

A %—area %
aq.—aqueous
Con.—concentrated
EtOAc/EA—ethyl acetate
DMA N,N-Dimethylacetamide
DIEA N,N-Diisopropylethylamine
TEA Triethylamine
THF Tetrahydrofuran
DCM Dichloromethane
2-MeTHF 2-Methyltetrahydrofuran
MeCN/CAN acetonitrile
MTBE methyl tert-butyl ether
iPrOAc Isopropyl acetate
eq.—equivalent
equip.—equipment
g—gram
GMP—good manufacturing practice
non—GMP—non good manufacturing practice
h—hours
HCl—hydrochloric acid
HSGC—headspace gas chromatography
IC—Ion chromatography
ICP-MS—Inductively coupled plasma mass spectrometry
IMP—impurity
IPC—in-process control
IR—Infrared absorption spectrum
KF—Karl Fischer (water determination)
kg—kilogram
L—liter
LCMS—Liquid Chromatography Mass Spectrometry
MBR—manufacturing batch record
mm—millimeter
N—mole
NaCl—Sodium chloride
NaHCO₃—Sodium bicarbonate
H₂SO₄—Sulfuric acid
N.D.—not detected
PLM—Polarized light microscopy
QC—Quality Control
Spec.—specification
STA—SynTheAll
H₂O—Water
a/a—Area by area
COA—Certificate of analysis
Eq.—equivalence
FIO—For information only
GC—Gas chromatography
HPLC—High performance liquid chromatography
NaOH—Sodium hydroxide
pH—hydrogen ion concentration
EtOH—ethanol
M.L./ML'S Mother liquor
min—minutes
mL—milli liter
mol—moles
NLT—Not less than
NMT—Not more than
PO—Purchase order
ppm—Parts per million
RRT—Relative retention time
RS—Residual Solvent
RT—Room temperature
STA—SynTheAll
Vol—Volumes
w/w—weight by weight
TP—Technique package

### Example 1: Preparation of gvcMMAE

### 1. Summary

The process for gvcMMAE has been well optimized, 36.53 g of product was obtained with 98.56 A % HPLC purity in 97.17% corrected yield. All test items met spec.

### 2. Introduction and Synthetic Scheme

For reaction step: The reaction condition was optimized as follow: equivalent of glutaric anhydride decreased from 1.2 eq. to 1.1 eq.; 5.2 eq. of DIEA was changed to 1.3 eq. of TEA; DMA/THF mix solvent was used as reaction solvent. In some studies the reaction temperature was decreased to 0° C. IPC of this condition was same as original condition but more stable and suitable for work up. In some studies the reaction temperature was increased to about 15-25° C.
For work up step: Reaction solution was directly charged into 75 v of THF/MTBE (1:2) solution and solid precipitated with good state.

The process has been well developed. For the system using TEA as base, a room reaction temperature was not tried since a clear solution was obtained at 0° C. . . . If the IPC is good and the reaction mixture is stable at RT, reacting at 0° C. might not be necessary.

### 3. Laboratory Work

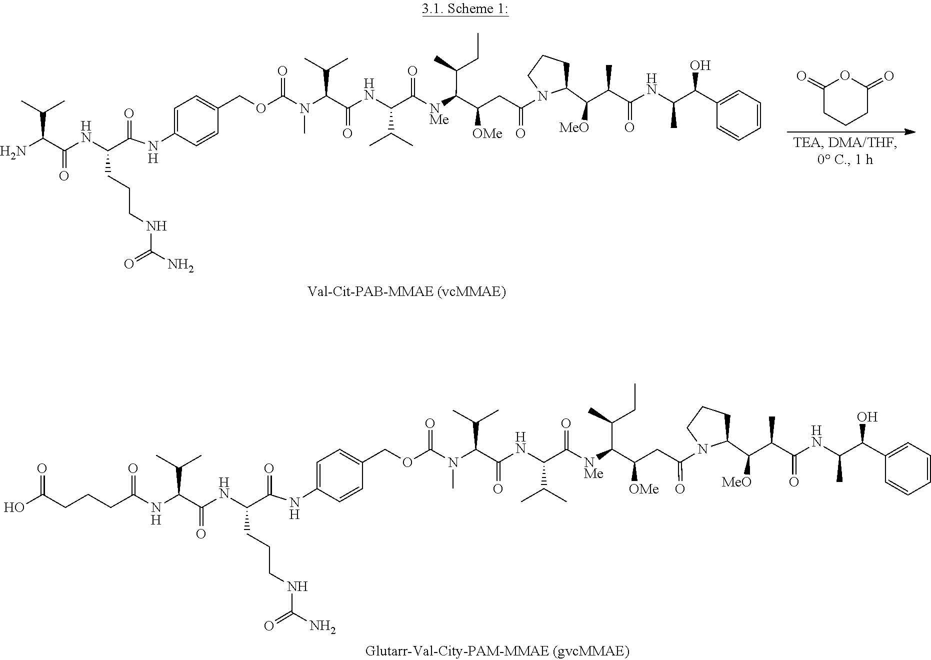 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; O=C1CCCC(=O)O1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)O)C(C)C)cc1)C(C)C]

### 3.2. Summary

In the original process, IPC purity was excellent but the reaction mixture proved unstable when held for a long time. Furthermore, the work-up stage of the original process was not suitable for scale-up because of the wall-sticking phenomenon. After process optimization, a more stable reaction condition was developed. The reaction solution was directly charged into anti-solvents (THF:MTBE=1:2, 75 v) and a good solid state was obtained. The new process is suitable for scale up.

### 3.3. Process Development

### 3.3.1 Familiar the TP Process (Table 1)

One reaction on 0.26 g scale of vcMMAE (assay corrected) was conducted to familiarize the process. An excellent 99.5 A % IPC purity was obtained after stirring for 1 h. However, the IPC purity decreased to 94.5 A % after stirring for 16 h. The result showed that the reaction mixture is not stable under these conditions. Reaction conditions (base/anhydride loading, reaction temperature) were further studied to solve the instability problem of the reaction. If the result proved repeatable and stable, the only concern would be how to isolate the solid state of the product.

Regarding the work up, after the charging reaction solution into acidic saturated brine solution (70 v), the solid precipitated but became sticky quickly. The result showed that DMA/brine is not a good crystallization system, so other solvents needed to be tried to find the best crystallization condition. As the high boiling point DMA is hard to remove when switching to other solvents, low boiling point solvents (DCM, MeCN, THF and 2-MeTHF) were targeted as reaction solvents to see if the same IPC result could be obtained. Besides, a lower DMA volume (3 v) was also to be tried if DMA proved the only choice for the reaction, because a lower volume of DMA could benefit the work-up process effectively (lower water for precipitation, lower organic solvent for extraction or precipitation).

TABLE 1
Preparation of gvcMMAE

| | | HPLC/area % | | | | | | | | | | | | | |
|---|---|---|---|---|---|---|---|---|---|---|---|---|---|---|---|
| | Temp./ | | RRT | RRT | RRT | | RRT | RRT | RRT | RRT | RRT | RRT | RRT | RRT | RRT |
| conditions | Time | RT | 0.90 | 0.96 | 0.98 | FP | 1.02 | 1.04 | 1.07 | 1.20 | 1.39 | 1.46 | 1.48 | 1.49 | 1.52 |
| vcMMAE: | 20~30° | IPC | 0.08 | 0.10 | 0.13 | 99.49 | 0.19 | — | — | — | — | — | — | — | — |
| 0.26 g | C., 1 h | 1 | | | | | | | | | | | | | |
| (0.3 g, | | | | | | | | | | | | | | | |
| assay: | 20~30° | IPC | 2.65 | 0.11 | 0.11 | 94.49 | 0.09 | 0.18 | 0.56 | 1.22 | 0.10 | 0.10 | 0.09 | 0.11 | 0.11 |
| 86.9%), | C., 16 h | 2 | | | | | | | | | | | | | |
| 1.0 eq. | | | | | | | | | | | | | | | |
| glutaric | | | | | | | | | | | | | | | |
| anhydride: | | | | | | | | | | | | | | | |
| 30.0 mg, | | | | | | | | | | | | | | | |
| 1.2 eq. | | | | | | | | | | | | | | | |
| DIEA: | | | | | | | | | | | | | | | |
| 0.14 g, | | | | | | | | | | | | | | | |
| 5.2 eq. | | | | | | | | | | | | | | | |
| DMA: 1.5 | | | | | | | | | | | | | | | |
| mL, 5 v | | | | | | | | | | | | | | | |

| | |
|---|---|
| Solid | Greasy solid formed |

### 3.3.2. Solvent Screening (Table 2 and 3)

Five different reactions solvents (DCM, MeCN, THF, 2-MeTHF and DMA/3 v) were tried. Among them, DMA gave the best result with 98.98 A % IPC purity. DCM and THF also gave good IPC purity of gvcMMAE. For MeCN as a solvent, a sticky solid was found during reaction. DCM and THF could be tried as alternative solvents for DMA if there proved no good precipitation method when using DMA. In addition, a mixed solvent system (DMA:THF=1:3, DMA:ACN=1:3) was also tried (after basic equivalent and temperature screening) and an acceptable IPC result was also obtained.

TABLE 2
Solvent screening.

| | | | HPLC/area % | |
|---|---|---|---|---|
| conditions | Temp./Time | | gvcMMAE | Note |
| vcMMAE: 0.3 g, 1.0 eq. (assay corrected). | 20~30° C., | 1 h | 97.93 | |
| Glutaric anhydride: 37.0 mg, 1.2 eq. | | | | |
| DIEA: 0.18 g, 5.2 eq. | | | | |
| DCM: 1.5 mL, 5 v | | | | |
| vcMMAE: 0.3 g, 1.0 eq. (assay corrected). | 20~30° C., | 1 h | 98.86 | sticky |
| Glutaric anhydride: 37.0 mg, 1.2 eq. | | | | |
| DIEA: 0.18 g, 5.2 eq. | | | | |
| MeCN: 1.5 mL, 5 v | | | | |
| vcMMAE: 0.3 g, 1.0 eq. (assay corrected). | 20~30° C., | 1 h | 98.04 | |
| Glutaric anhydride: 37.0 mg, 1.2 eq. | | | | |
| DIEA: 0.18 g, 5.2 eq. | | | | |
| THF: 1.5 mL, 5 v | | | | |
| vcMMAE: 0.3 g, 1.0 eq. (assay corrected). | 20~30° C., | 1 h | 67.85 | |
| Glutaric anhydride: 37.0 mg, 1.2 eq. | | | | |
| DIEA: 0.18 g, 5.2 eq. | | | | |
| 2-MeTHF: 1.5 mL, 5 v | | | | |
| vcMMAE: 0.3 g, 1.0 eq. (assay corrected). | 20~30° C., | 1 h | 98.98 | |
| Glutaric anhydride: 37.0 mg, 1.2 eq. | 20~30° C., | 16 h | 98.08 | |
| DIEA: 0.18 g, 5.2 eq. | | | | |
| DMA: 0.9 mL, 3 v | | | | |
| vcMMAE: 0.3 g, 1.0 eq. (assay corrected). | −5-5° C., | 1 h | 99.23 | |
| Glutaric anhydride: 37.0 mg, 1.2 eq. | | | | |
| DIEA: 0.046 g, 1.3 eq. | | | | |
| DMA: 0.3 mL, 1 v | | | | |
| THF: 0.9 mL, 3 v | | | | |
| vcMMAE: 0.3 g, 1.0 eq. (assay corrected). | −5-5° C., | 1 h | 98.90 | |
| Glutaric anhydride: 37.0 mg, 1.2 eq. | | | | |
| DIEA: 0.046 g, 1.3 eq. | | | | |
| DMA: 0.3 mL, 1 v | | | | |
| ACN: 0.9 mL, 3 v | | | | |

TABLE 3
Impurity profile of gvcMMAE

| | HPLC/area % | | | | | | | | | | | | |
|---|---|---|---|---|---|---|---|---|---|---|---|---|---|
| | RRT | RRT | RRT | RRT | RRT | | RRT | RRT | RRT | RRT | RRT | RRT | RRT |
| Solvents | 0.83 | 0.90 | 0.94 | 0.96 | 0.98 | gvcMMAE | 1.02 | 1.04 | 1.05 | 1.12 | 1.28 | 1.29 | 1.31 |
| DMA, 5 v | — | 0.08 | — | 0.10 | 0.13 | 99.49 | 0.19 | — | — | — | — | — | — |
| DCM, 5 v | 0.33 | 0.40 | 0.02 | 0.11 | 0.13 | 97.93 | 0.17 | 0.09 | — | — | 0.04 | 0.04 | 0.62 |
| MeCN, 5 v | | 0.40 | | 0.08 | 0.13 | 98.86 | 0.17 | 0.12 | — | — | 0.09 | — | 0.16 |
| THF, 5 v | | 0.06 | 0.05 | — | 0.13 | 98.04 | 0.17 | 0.06 | 0.09 | — | 0.03 | 0.11 | 0.86 |
| 2-MeTHF 5 v | | | | | | 67.85 | — | — | 24.37 | 2.00 | 5.78 | | |
| DMA, 3 v | | 0.08 | | 0.10 | 0.14 | 98.98 | 0.18 | 0.09 | — | — | — | | 0.26 |
| | | 1.14 | | 0.10 | 0.10 | 98.10 | 0.06 | 0.08 | | | | | 0.26 |
| DMA:THF = | — | — | 0.06 | 0.11 | — | 99.23 | 0.16 | 0.06 | | | | | 0.38 |
| 1:3 | | | | | | | | | | | | | |
| DMA:ACN = | | | 0.06 | | 0.32 | 98.90 | 0.12 | 0.10 | | | | | 0.49 |
| 1:3 | | | | | | | | | | | | | |

### 3.3.3. Base Equivalent and Temperature Screening (Table 4 and 5)

3 reactions with different equivalents of base (1.3 eq., 2.4 eq. and 5.2 eq.) were carried out at low temperature (−5~5° C.). Results showed that 1.3 eq. of DIEA was sufficient to achieve the complete reaction. By extending the reaction time to 16 h, the IPC purity decreased only slightly, indicating that the reaction is sufficiently stable at −5~5° C. for 16 h.

The reactions with DCM and THF solvents were also tried at −5~5° C. IPC purity was still relatively high and the mixture also had good stability at low reaction temperature. −5~5° C. and 1 h could be defined for the reaction.

TABLE 4

| | | HPLC/area % |
|---|---|---|
| conditions | Temp./Time | gvcMMAE |
| vcMMAE: 0.3 g, 1.0 eq. | −5~5° C./1 h | 99.10 |
| (assay corrected). | −5~5° C./16 h | 99.03 |
| Glutaric anhydride: 37.0 mg, 1.2 eq. | | |
| DIEA: 0.046 g, 1.3 eq. | | |
| DMA: 0.9 mL, 3 v | | |
| vcMMAE: 0.3 g, 1.0 eq. | −5~5° C./1 h | 99.15 |
| (assay corrected). | −5~5° C./16 h | 98.98 |
| Glutaric anhydride: 37.0 mg, 1.2 eq. | | |
| DIEA: 0.084 g, 2.4 eq. | | |
| DMA: 0.9 mL, 3 v | | |
| vcMMAE: 0.3 g, 1.0 eq. | −5~5° C./1 h | 99.04 |
| (assay corrected). | −5~5° C./16 h | 98.84 |
| Glutaric anhydride: 37.0 mg, 1.2 eq. | | |
| DIEA: 0.18 g, 5.2 eq. | | |
| DMA: 0.9 mL, 3 v | | |
| vcMMAE: 0.3 g, 1.0 eq. | −5~5° C./1 h | 98.44 |
| (assay corrected). | −5~5° C./16 h | 98.20 |
| Glutaric anhydride: 37.0 mg, 1.2 eq. | | |
| DIEA: 0.046 g, 1.3 eq. | | |
| DCM: 0.9 mL, 3 v | | |
| vcMMAE: 0.3 g, 1.0 eq. | −5~5° C./1 h | 98.47 |
| (assay corrected). | −5~5° C./16 h | 98.34 |
| Glutaric anhydride: 34.0 mg, 1.2 eq. | | |
| DIEA: 0.046 g, 1.3 eq. | | |
| THF: 0.9 mL, 3 v | | |

TABLE 5
Impurity profile of gvcMMAE

| | HPLC/area % | | | | | | | | | | | | |
|---|---|---|---|---|---|---|---|---|---|---|---|---|---|
| | RRT | RRT | RRT | RRT | RRT | | RRT | RRT | RRT | RRT | RRT | RRT | RRT |
| Conditions | 0.83 | 0.90 | 0.94 | 0.96 | 0.98 | gvcMMAE | 1.02 | 1.03 | 1.04 | 1.19 | 1.28 | 1.31 | 1.34 |
| DMA, 3 v | — | — | 0.06 | 0.11 | 0.13 | 99.10 | 0.18 | | 0.09 | — | 0.03 | 0.24 | — |
| DIEA: 1.3 | — | 0.05 | 0.06 | 0.11 | 0.13 | 99.03 | 0.18 | 0.06 | 0.10 | — | 0.03 | 0.25 | — |
| eq. −5~5° C. | | | | | | | | | | | | | |
| DMA, 3 v | — | — | 0.06 | 0.11 | 0.13 | 99.15 | 0.17 | — | 0.08 | — | — | 0.25 | — |
| DIEA: 2.4 | — | 0.09 | 0.06 | 0.11 | 0.13 | 98.97 | 0.18 | 0.06 | 0.10 | — | 0.04 | 0.26 | — |
| eq. −5~5° C. | | | | | | | | | | | | | |
| DMA, 3 v | — | 0.03 | 0.05 | 0.11 | 0.13 | 99.04 | 0.19 | 0.06 | 0.09 | — | 0.04 | 0.27 | — |
| DIEA: 5.2 | — | 0.20 | 0.05 | 0.11 | 0.13 | 98.84 | 0.18 | 0.07 | 0.10 | — | 0.05 | 0.27 | — |
| eq. −5~5° C. | | | | | | | | | | | | | |
| DCM, 3 v | 0.08 | 0.21 | 0.05 | 0.11 | 0.12 | 98.44 | 0.18 | 0.06 | 0.09 | — | — | 0.66 | — |
| DIEA: 1.3 | — | 0.62 | — | 0.11 | 0.13 | 98.20 | 0.15 | — | 0.08 | 0.07 | — | 0.63 | — |
| eq. −5~5° C. | | | | | | | | | | | | | |
| THF, 3 v | — | 0.05 | 0.06 | 0.11 | 0.12 | 98.47 | 0.17 | 0.06 | 0.09 | — | 0.04 | 0.79 | 0.05 |
| DIEA: 1.3 | — | 0.32 | — | 0.11 | 0.12 | 98.34 | 0.17 | — | 0.08 | 0.08 | — | 0.77 | — |
| eq. −5~5° C. | | | | | | | | | | | | | |

### 3.3.4. Screen the Loading of Glutaric Anhydride (Table 6 and 7)

Different equivalents of glutaric anhydride (1.05 eq. and 1.10 eq.) were screened. 1.10 eq. glutaric anhydride is enough for full conversion of the reaction.

TABLE 6

| | | HPLC/area % |
|---|---|---|
| conditions | Temp./Time | gvcMMAE |
| vcMMAE: 0.3 g, 1.0 eq. (assay corrected). | −5-5° C., 1 h | 98.9% |
| Glutaric anhydride: 29.8 mg, 1.05 eq. | | |
| DIEA: 0.046 g, 1.3 eq. | | |
| DMA: 0.3 mL, 1 v | | |
| THF: 0.9 mL, 3 v | | |
| vcMMAE: 0.3 g, 1.0 eq. (assay corrected). | −5-5° C., 1 h | 98.7% |
| Glutaric anhydride: 31.7 mg, 1.10 eq. | | |
| DIEA: 0.046 g, 1.3 eq. | | |
| DMA: 0.3 mL, 1 v | | |
| THF: 0.9 mL, 3 v | | |

TABLE 7

| | HPLC/area % | | | | | | | | | | |
|---|---|---|---|---|---|---|---|---|---|---|---|
| | | | | | | | | | | vcMMAE | |
| | RRT | RRT | RRT | RRT | | RRT | RRT | RRT | RRT | RRT | RRT |
| Conditions | 0.90 | 0.94 | 0.96 | 0.98 | gvcMMAE | 1.02 | 1.04 | 1.05 | 1.20 | 1.22 | 1.31 |
| 1.05 eq. | | 0.06 | 0.13 | 0.20 | 98.74 | 0.16 | 0.12 | | | 0.17 | 0.42 |
| glutaric | | | | | | | | | | | |
| anhydride | | | | | | | | | | | |
| 1.1 eq. | | 0.05 | 0.13 | 0.23 | 98.90 | 0.15 | 0.14 | | | | 0.40 |
| glutaric | | | | | | | | | | | |
| anhydride | | | | | | | | | | | |

3.3.5. 1^{st} round process optimization (Table 8 and 9)

A small change to the process was developed using a DMA/THF mixed solvent system and sulfuric/5% Na₂SO₄ solution quenching system.

One batch on 5 g scale of vcMMAE was conducted to verify this process. After reaction, IPC purity was 99.37 A %. The reaction solution was inversely added to 30 v of sulfuric (1.5 eq.)/5% Na₂SO₄ solution at −5-5° C. After filtering and drying, 7.0 g of crude product (contains Na₂SO₄) was obtained with 98.71 A % HPLC purity. ~6.36 g of crude gvcMMAE was further made as a slurry in 30 v of water to remove Na₂SO₄. After filtering and drying, 4.2 g of gvcMMAE was obtained with 98.89 A % HPLC purity in 84% crude yield.

IC results showed that residual SO₄²⁻ in gvcMMAE was 0.70%, which means the product is free acid state. Residual Na⁺ was 0.37%. X-RPD showed that it might be amorphous.

The DVS test showed that the product is hygroscopic at 80% RH at 25° C. (Uptake 3.6% water on sorption curve between 40% RH and 80% RH, at 25° C.).

TABLE 8

| | | | | HPLC/area % | |
|---|---|---|---|---|---|
| Input | conditions | Temp./Time | | gvcMMAE | Note |
| 5.0 g | vcMMAE: 5.0 g, 1.0 eq. | −5-5° C., | 1 h | 99.37 | |
| | (5.75 g, assay: 86.9%). | | | | |
| | Glutaric anhydride: | | | | |
| | 610 mg, 1.2 eq. | | | | |
| | DIEA: 750 mg, 1.3 eq. | | | | |
| | DMA: 5 mL, 1 v | | | | |
| | THF: 15 mL, 3 v | | | | |
| 5.0 g scale | vcMMAE: 5.0 g scale, | −5-5° C., | 1 h | 98.71 | 20-25° C., under |
| reaction | DMA/THF solution | | | | vacuum, drying |
| solution (from | sulfuric acid (1.5 eq.)/5% | | | | for 16 hr |
| above)) | sodium sulfate solution: | | | | Solid state: powder |
| | 150 mL, 30 v | | | | |
| | −5-5° C., reverse adding | | | | |
| 6.36 g crude | Crude gvcMMAE: 6.36 g | 20-30° C., | 1 h | 98.89 | 20-25° C., under |
| gvcMMAE | (contain Na₂SO₄) | | | | vacuum, drying |
| (from above) | Water: 150 mL, 30 v | | | | for 16 hr |
| | | | | | Solid state: powder |

TABLE 9

| | HPLC/area % | | | | | | | | | | |
|---|---|---|---|---|---|---|---|---|---|---|---|
| | RRT | RRT | RRT | RRT | | RRT | RRT | RRT | RRT | RRT | RRT |
| Conditions | 0.90 | 0.94 | 0.96 | 0.98 | gvcMMAE | 1.02 | 1.04 | 1.05 | 1.20 | 1.22 | 1.31 |
| DMA:THF = | | 0.05 | 0.14 | | 99.37 | | 0.08 | | | | 0.36 |
| 1:3 | | | | | | | | | | | |
| Work up | | 0.06 | 0.05 | 0.42 | 98.71 | 0.17 | 0.16 | | | 0.07 | 0.36 |
| 5% Na₂SO₄ | | | | | | | | | | | |
| Work up | | 0.06 | 0.09 | 0.25 | 98.89 | 0.14 | 0.13 | | | | 0.39 |
| 30 v water | | | | | | | | | | | |

3.3.6. Study the Solubility of gvcMMAE in Different Solvents (Table 10)

Solubility of gvcMMAE (free acid state) in different solvents (THF, DCM, MTBE, n-Heptane, iPrOAc, 1,4-dioxane) were investigated. According to the assay results, the free base state of gvcMMAE is insoluble in most solvents.

TABLE 10

| conditions | Temp./Time | Assay in ML (w/w %) |
|---|---|---|
| gvcMMAE: 0.05 g | 20-30° C., 0.5 h | 0.47 |
| THF: 1 mL, 20 v | | |
| gvcMMAE: 0.05 g | 20-30° C., 0.5 h | 0.00 |
| DCM: 1 mL, 20 v | | |
| gvcMMAE: 0.05 g | 20-30° C., 0.5 h | 0.00 |
| MTBE: 1 mL, 20 v | | |
| gvcMMAE: 0.05 g | 20-30° C., 0.5 h | 0.00 |
| N-heptane: 1 mL, 20 v | | |
| gvcMMAE: 0.05 g | 20-30° C., 0.5 h | 0.00 |
| iPrOAc: 1 mL, 20 v | | |
| gvcMMAE: 0.05 g | 20-30° C., 0.5 h | 0.22 |
| 1,4-dioxane: 1 mL, 20 v | | |

### 3.3.7. Study the Salt Formation of Free Acid (Table 11)

Five bases (Calcium hydroxide, Dicyclohexylamine, DABCO, Tributylamine and Barium hydroxide) were tried for salt formation of gvcMMAE; THF was used as solvent. For dicyclohexylamine and tributylamine systems, the product was like milk after adding base, and the purity of the mixture decreased to ~96 A % and a 2 A % impurity @ RRT1.76 was observed, (the mixture was concentrated to ~30 v and 60 v of MTBE added, whence a solid precipitated. The purity of wet solid precipitated from THF/MTBE/tributylamine system was 98.87 A %, assay in ML was 0.01%. It was considered worth trying to get solid by adding MTBE into THE/TEA or THF/tributylamine reaction solution.) For the DABCO system, the product dissolved quickly but became a jelly after stirring for 0.5 h, and the purity of the mixture decreased significantly. For the Barium hydroxide system, the solid precipitated after stirring for 1 h, and the purity of wet cake decreased to 96.32 A %. For Calcium hydroxide system, the product was like milk after stirring for 1 h.

According to the phenomenon, Tributylamine and TEA (similar properties) were considered as base for the reaction.

TABLE 11

| | | HPLC purity (area %) | | | |
|---|---|---|---|---|---|
| conditions | Temp./Time | RRT0.52 | FP | RRT1.76 | Note |
| Solid before salt formation | / | 0 | 98.50 | 0.07 | |
| gvcMMAE: 0.1 g | 20-30° C., 0.5 h | 0.32 | 96.2 | 2.16 | like milk |
| THF: 6 mL, 60 v | | | | | |
| Dicyclohexylamine: 8.1 mg, | | | | | |
| 1.0 eq. | | | | | |
| Concentrate to ~30 v, add | 20-30° C., 0.5 h | 0 | 97.72 | 0.07 | Solid precipitated |
| 60 v of MTBE | | | | | |
| gvcMMAE: 0.1 g | 20-30° C., 0.5 h | 0.65 | 95.5 | 2.23 | like milk |
| THF: 6 mL, 60 v | | | | | |
| Tributylamine: 15.0 mg, 1.0 | | | | | |
| eq. | | | | | |
| Concentrate to ~30 v, add | 20-30° C., 0.5 h | 0 | 98.87 | 0.04 | Solid precipitated |
| 60 v of MTBE | | | | | Assay in ML: |
| | | | | | 0.01% |
| gvcMMAE: 0.1 g | 20-30° C., 0.5 h | | 53.4 | | Clear, but |
| THF: 6 mL, 60 v | | | | | became jelly |
| DABCO: 10.0 mg, 1.0 eq. | | | | | quickly |
| gvcMMAE: 0.1 g | 20-30° C., 1 h | — | — | — | like milk, no solid |
| THF: 6 mL, 60 v, Water: 2 | | | | | after filtration |
| drops for dissolution- | | | | | |
| promotion | | | | | |
| Calcium hydroxide: 4.0 mg, | | | | | |
| 0.58 eq. | | | | | |
| gvcMMAE: 0.1 g | 20-30° C., 1 h | 0 | 96.32 | 0.08 | Solid precipitated |
| THF: 6 mL, 60 v, Water: 2 | | | | | |
| drops for dissolution- | | | | | |
| promotion | | | | | |
| Barium hydroxide: 8.0 mg, | | | | | |
| 0.67 eq. | | | | | |

### 3.3.8. Study the Salt Formation by Using Tributylamine or TEA (Table 12 and 13)

Four reactions on 0.3 g scale of vcMMAE were carried out with different base/solvent system (base: tributylamine, TEA; solvent: THF, THF/DMA mix solvent) to study the separation of gvcMMAE salt.

For a single solvent system (with THE as solvent), the reaction solution became colloidal quickly after adding base (tributylamine as base: turbid colloidal; TEA as base: transparent colloid).

For THF/DMA/tributylamine system, the reaction solution is clear. Then, 30 v of THF was dropped into the reaction solution for dilution, and the system became a jelly (milder than single solvent).

For THF/DMA/TEA system, the reaction solution is clear. When 30 v of THF was dropped into the reaction solution and the solvent became thick, then became turbid. When 60 v of MTBE was added into the mixture, a solid precipitated. After filtration, 0.275 g of solid was obtained with 98.64 A % HPLC purity in 78.4% crude yield.

THF/DMA/TEA reaction system and THF/MTBE work up system were investigated further.

TABLE 12

| | | | | HPLC purity | |
|---|---|---|---|---|---|
| Input | conditions | Temp./Time | | (area %) | Note |
| 0.3 g vcMMAE | vcMMAE: 0.3 g, 1.0 eq. (assay corrected). | −5-5° C., | 1 h | gvcMMAE: | Clear |
| | Glutaric anhydride: 32.0 mg, 1.05 eq. | | | 98.44% | solution |
| | TEA: 36 mg, 1.3 eq. | | | | |
| | DMA: 0.3 mL, 1 v | | | | |
| | THF: 0.9 mL, 3 v | | | | |
| Work up | THF: 9 mL, 30 v | 20-30° C., | 5 min | gvcMMAE: | Turbid |
| | | | | 98.64% | solution |
| | MTBE: 18 mL, 60 v | 20-30° C., | 2 h | Wet cake | Suspension |
| 0.3 g vcMMAE | vcMMAE: 0.3 g, 1.0 eq. (assay corrected). | 20-30° C., | 1 h | gvcMMAE: | Transparent |
| | Glutaric anhydride: 32.0 mg, 1.05 eq. | | | 95.95% (mix | colloid, hard |
| | TEA: 36 mg, 1.3 eq. | | | sample, | to stir |
| | THF: 1.2 mL, 4 v | | | jelly) | |
| 0.3 g vcMMAE | vcMMAE: 0.3 g, 1.0 eq. (assay corrected). | −5-5° C., | 1 h | gvcMMAE: | Clear |
| | Glutaric anhydride: 32.0 mg, 1.05 eq. | | | 98.29% | solution |
| | tributylamine: 65 mg, 1.3 eq. | | | | |
| | DMA: 0.3 mL, 1 v | | | | |
| | THF: 0.9 mL, 3 v | | | | |
| Work up | THF: 9 mL, 30 v | 20-30° C., | 5 min | — | Jelly |
| 0.3 g vcMMAE | vcMMAE: 0.3 g, 1.0 eq. (assay corrected). | 20-30° C., | 1 h | gvcMMAE: | Turbid |
| | Glutaric anhydride: 32.0 mg, 1.05 eq. | | | 96.62% | colloidal |
| | tributylamine: 65 mg, 1.3 eq. | | | | hard to stir |
| | THF: 1.2 mL, 4 v | | | | |

TABLE 13

| | HPLC/area % | | | | | | | | | | | | |
|---|---|---|---|---|---|---|---|---|---|---|---|---|---|
| | | | | | | | | | | RRT | | | |
| | | RRT | RRT | RRT | RRT | | RRT | RRT | RRT | 1.08 | RRT | RRT | RRT |
| Conditions | Status | 0.90 | 0.94 | 0.96 | 0.98 | gvcMMAE | 1.02 | 1.04 | 1.05 | vcMMAE | 1.20 | 1.31 | 1.78 |
| DMA/THF/TEA | IPC | — | 0.07 | 0.12 | 0.14 | 98.44 | 0.16 | 0.04 | 0.08 | 0.42 | — | 0.45 | 0.07 |
| | Solid | — | 0.06 | 0.13 | 0.14 | 98.64 | 0.15 | 0.05 | 0.08 | — | — | 0.43 | — |
| THF/TEA | MIX | — | 0.06 | 0.15 | 0.13 | 95.95 | 0.16 | 0.06 | 0.09 | 0.25 | 0.06 | 1.02 | 1.42 |
| | jelly | | | | | | | | | | | | |
| DMA/THF/ | IPC | — | 0.07 | 0.14 | 0.15 | 98.29 | 0.16 | 0.06 | 0.08 | 0.05 | 0.02 | 0.57 | 0.16 |
| tributylamine | | | | | | | | | | | | | |
| THF/ | MIX | — | 0.10 | 0.16 | 0.13 | 96.62 | 0.16 | 0.06 | 0.08 | 0.06 | 0.06 | 0.76 | 1.15 |
| tributylamine | Jelly | | | | | | | | | | | | |

### 3.3.9. Study the Ratio of DMA to Benefit the Work Up Stage (Table 14 and 15)

Three reactions with different ratios of DMA:THF were conducted on a 0.3 g scale of vcMMAE to study the suitable work up conditions of for isolation of the TEA salt. A good IPC result (99.14 A %) can be obtained when DMA:THF=0.25 v:3.75 v

For the original reaction conditions (DMA:THF=1:3), when 30 v of THF was added directly into the reaction solution, the solution became thick with a risk of colloid, which is not suitable for scale up. Reverse addition of the reaction solution into solvent was therefore considered.

Consequently, the other two reaction solutions (DMA:THF=0.5:3.5 and DMA:THF=0.25:3.75) were dropped into 90 v (30 v THF+60 v MTBE) of previously mixed solvents, and a good state solid precipitated.

TABLE 14

| | | | | | HPLC | |
|---|---|---|---|---|---|---|
| Lot/Batch | | | | | purity | |
| No. | Input | conditions | Temp./Time | | (area %) | Note |
| 1 | 0.3 g vcMMAE | vcMMAE: 0.3 g, 1.0 eq. | −5-5° C., | 1 h | gvcMMAE: | Clear |
| | | (assay corrected). | | | 98.95% | solution |
| | | Glutaric anhydride: | | | | |
| | | 34.0 mg, 1.10 eq. | | | | |
| | | TEA: 36 mg, 1.3 eq. | | | | |
| | | DMA: 0.3 mL, 1 v | | | | |
| | | THF: 0.9 mL, 3 v | | | | |
| | Work up | THF: 9 mL, 30 v | 20-30° C., | 1 h | gvcMMAE: | Thick with |
| | | | | | 98.50% | a risk of |
| | | | | | | colloid |
| | | MTBE: 18 mL, 60 v | 20-30° C., | 2 h | | Solid |
| | | | | | | precipitated |
| 2 | 0.3 g vcMMAE | vcMMAE: 0.3 g, 1.0 eq. | −5-5° C., | 1 h | gvcMMAE: | Clear |
| | | (assay corrected). | | | 98.86% | solution |
| | | Glutaric anhydride: | | | | |
| | | 34.0 mg, 1.10 eq. | | | | |
| | | TEA: 36 mg, 1.3 eq. | | | | |
| | | DMA: 0.15 mL, 0.5 v | | | | |
| | | THF: 1.05 mL, 3.5 v | | | | |
| | Work up | Reverse adding all of | 20-30° C., | 0.5 h | gvcMMAE: | Stickier |
| | | reaction solution | | | 98.99% | than half |
| | | THF: 4.5 mL, 15 v | | | | adding |
| | | MTBE: 9.0 mL, 30 v | | | | |
| 3 | 0.3 g vcMMAE | vcMMAE: 0.3 g, 1.0 eq. | −5-5° C., | 1 h | gvcMMAE: | Clear |
| | | (assay corrected). | | | 99.14% | solution |
| | | Glutaric anhydride: | | | | (thick at −5-5° |
| | | 34.0 mg, 1.10 eq. | | | | C., clear |
| | | TEA: 36 mg, 1.3 eq. | | | | at 20-30° C.) |
| | | DMA: 0.075 mL, 0.25 v | | | | |
| | | THF: 1.125 mL, 3.75 v | | | | |
| | Work up | Reverse adding all of | 20-30° C., | 1 h | gvcMMAE: | Solid with |
| | | reaction solution | | | 98.36% | good state |
| | | THF: 9.0 mL, 30 v | | | | (better than |
| | | MTBE: 18.0 mL, 60 v | | | | entry 2) |

TABLE 15

| | | HPLC/area % | | | | | | | | | | | | |
|---|---|---|---|---|---|---|---|---|---|---|---|---|---|---|
| Lot/ | | | | | | | | | | | RRT | | | |
| Batch | | | RRT | RRT | RRT | RRT | | RRT | RRT | RRT | 1.08 | RRT | RRT | RRT |
| No. | Conditions | Status | 0.90 | 0.94 | 0.96 | 0.98 | gvcMMAE | 1.02 | 1.04 | 1.05 | vcMMAE | 1.20 | 1.31 | 1.78 |
| 1 | DMA:THF = 1:3 | IPC | — | 0.05 | 0.13 | 0.14 | 98.95 | 0.18 | 0.06 | 0.07 | 0.01 | — | 0.37 | — |
| | positive adding | Solid | | 0.9 | 0.15 | 0.27 | 98.50 | 0.17 | 0.14 | — | — | — | 0.47 | — |
| 2 | DMA:THF = 0.5:3.5 | IPC | — | 0.05 | 0.11 | 0.15 | 98.86 | 0.16 | 0.04 | 0.08 | 0.01 | — | 0.46 | — |
| | reverse adding | WET | — | 0.04 | 0.10 | 0.11 | 98.99 | 0.13 | 0.04 | 0.06 | 0.03 | — | 0.47 | — |
| 3 | DMA:THF = 0.25:3.75 | IPC | — | 0.04 | 0.09 | 0.11 | 99.14 | 0.05 | 0.02 | 0.05 | — | — | 0.45 | — |
| | reverse adding | WET | — | 0.04 | 0.13 | 0.15 | 98.36 | 0.14 | 0.06 | 0.09 | — | — | 0.60 | |

### 3.3.10 Study the Volume and Ratio of THF/MTBE (Table 16 and 17)

Three reactions were conducted on a 1.0 g scale of vcMMAE to study the volume of antisolvents (THF:MTBE=1:2; 90 v, 75 v and 60 v were tried). After filtration and vacuum drying, 1.1 g of gvcMMAE TEA salt (90 v work up batch) with 98.98 A % HPLC purity with 92.4% crude yield. (Residual solvents by HSGC: THF: 0.83%, MTBE: 5.23%, DMA: 1.35%; Residual TEA by IC: 1.8%).

100 mg of gvcMMAE was stood at 60% RH condition to do a moisture absorption experiment. The solid state had no change (The weight changed to 103 mg) after 3 days.

Different ratios of THF/MTBE were also tried (THF:MTBE=1:3, THF:MTBE=1:4, 45 v and 60 v), but no better results were observed. The solid stuck to the wall after extending the stirring time (16 hr).

Considering both volume and solid state, 75 v of THF/MTBE was determined to be the best option.

TABLE 16

| | | | | | HPLC | |
|---|---|---|---|---|---|---|
| | | | Temp./ | | purity | |
| Entry | Input | conditions | Time | Output | (area %) | Note |
| 1 | gvcMMAE | Reverse adding all of | 20-30° C., | 1.1 g Dry | gvcMMAE: | Solid with |
| | reaction solution | reaction solution | 1 h | solid | 98.49% | good state |
| | (1.0 g scale, | THF: 30 mL, 30 v | | | | (better than |
| | DMA:THF = | MTBE: 60 mL, 60 v | | | | entry 2&3) |
| | 0.25:3.75) | | | | | |
| 2 | gvcMMAE | Reverse adding all of | 20-30° C., | 1.1 g Dry | gvcMMAE: | Solid with |
| | reaction solution | reaction solution | 1 h | solid | 98.33% | good state |
| | (1.0 g scale, | THF: 25 mL, 25 v | | | | (better than |
| | DMA:THF = | MTBE: 50 mL, 50 v | | | | entry 3) |
| | 0.25:3.75) | | | | | |
| 3 | gvcMMAE | Reverse adding all of | 20-30° C., | 1.1 g Dry | gvcMMAE: | Solid, stickier |
| | reaction solution | reaction solution | 1 h | solid | 98.18% | than entry 2 |
| | (1.0 g scale, | THF: 20 mL, 20 v | | | | (A little solid |
| | DMA:THF = | MTBE: 40 mL, 40 v | | | | sinks to the |
| | 0.25:3.75) | | | | | bottom of the |
| | | | | | | bottle after |
| | | | | | | filtration) |

TABLE 17

| | | HPLC/area % | | | | | | | | | | | | |
|---|---|---|---|---|---|---|---|---|---|---|---|---|---|---|
| | | | | | | | | | | | RRT | | | |
| | | | RRT | RRT | RRT | RRT | | RRT | RRT | RRT | 1.08 | RRT | RRT | RRT |
| Entry | Conditions | Status | 0.90 | 0.94 | 0.96 | 0.98 | gvcMMAE | 1.02 | 1.04 | 1.05 | vcMMAE | 1.20 | 1.31 | 1.78 |
| 1 | 90 v | WET | | 0.02 | 0.11 | 0.06 | 98.98 | 0.13 | 0.09 | | | | 0.62 | |
| | THF/MTBE, | Solid | — | 0.05 | 0.10 | 0.32 | 98.49 | 0.18 | 0.16 | — | — | — | 0.64 | — |
| | Reverse adding | | | | | | | | | | | | | |
| 2 | 75 v | Solid | — | 0.05 | 0.05 | 0.22 | 98.33 | 0.13 | 0.14 | — | — | — | 0.68 | — |
| | THF/MTBE, | | | | | | | | | | | | | |
| | Reverse adding | | | | | | | | | | | | | |
| 3 | 60 v | Solid | — | 0.04 | 0.06 | 0.25 | 98.18 | 0.15 | 0.13 | — | — | — | 0.70 | — |
| | THF/MTBE, | | | | | | | | | | | | | |
| | Reverse adding | | | | | | | | | | | | | |

### 3.3.11. Study the Work Up at Low Temperature (Table 18 and 19)

Four batches of the work up were carried out with different volumes of antisolvent (THF:MTBE=1:2; 30 v, 45 v, 60 v, 75 v) at −5~5° C. The 75 v/−5~5° C. condition gave the best result with good solid state, which did not stick to the wall after extending the stirring time (16 h).

TABLE 18

| | | | | HPLC | |
|---|---|---|---|---|---|
| | | | | purity | |
| Entry | Input | conditions | Temp./Time | (area %) | Note |
| 1 | gvcMMAE | THF: 5 mL, 10 v | −5~5° C., 1 h | gvcMMAE: | Stick to the wall |
| | reaction solution | MTBE: 10 mL, 20 v | | 98.46% | after stirring for 4 |
| | (0.5 g scale, | | | | hr. |
| | DMA:THF = | | | | |
| | 0.25:3.75) | | | | |
| 2 | gvcMMAE | THF: 7.5 mL, 15 v | −5~5° C., 1 h | gvcMMAE: | Good solid state |
| | reaction solution | MTBE: 15 mL, 30 v | | 98.49% | after stirring for 4 |
| | (0.5 g scale, | | | | hr, but stick to the |
| | DMA:THF = | | | | wall after stirring |
| | 0.25:3.75) | | | | for 16 hr. |
| 3 | gvcMMAE | THF: 10 mL, 20 v | −5~5° C., 1 h | gvcMMAE: | Good solid state |
| | reaction solution | MTBE: 20 mL, 40 v | | 98.29% | after stirring for 4 |
| | (0.5 g scale, | | | | hr, but stick to the |
| | DMA:THF = | | | | wall after stirring |
| | 0.25:3.75) | | | | for 16 hr. |
| 4 | gvcMMAE | THF: 12.5 mL, 25 v | −5~5° C., 1 h | gvcMMAE: | Good solid state |
| | reaction solution | MTBE: 25 mL, 50 v | | 98.56% | after stirring for 16 |
| | (0.5 g scale, | | | | hr, |
| | DMA:THF = | | | | |
| | 0.25:3.75) | | | | |

TABLE 19

| | HPLC/area % | | | | | | | | | | | | |
|---|---|---|---|---|---|---|---|---|---|---|---|---|---|
| | | | | | | | | | | RRT | | | |
| | | RRT | RRT | RRT | RRT | | RRT | RRT | RRT | 1.08 | RRT | RRT | RRT |
| Conditions | Status | 0.90 | 0.94 | 0.96 | 0.98 | gvcMMAE | 1.02 | 1.04 | 1.05 | vcMMAE | 1.20 | 1.31 | 1.78 |
| −5~5° C., 30 v | WET | 0.07 | 0.04 | 0.13 | 0.31 | 98.46 | 0.18 | 0.15 | — | — | — | 0.56 | — |
| −5~5° C., 45 v | WET | 0.02 | 0.04 | 0.14 | 0.32 | 98.49 | 0.18 | 0.15 | — | — | — | 0.58 | — |
| −5~5° C., 60 v | WET | — | 0.06 | 0.13 | 0.48 | 98.29 | 0.18 | 0.15 | — | — | — | 0.58 | — |
| −5~5° C., 75 v | WET | 0.04 | 0.05 | 0.11 | 0.32 | 98.56 | 0.14 | 0.15 | — | — | — | 0.57 | — |

3.3.12. Demo Conjugation Study for gvcMMAE and Peptide (Table 20 and 21)

Two demo reactions were conducted on 0.1 g scale of peptide (BCY8234, 93.76% HPLC purity). For gvcMMAE prepared from sulfuric/5% Na₂SO₄ solution, the IPC purity was 80.34%. After work up, the purity of wet cake was 80.36%; For gvcMMAE prepared from TEA condition, the IPC purity was 86.51%. After work up, the purity of wet cake was 86.25%. The result showed that gvcMMAE from TEA solution gave better IPC purity.

TABLE 20

| | | | | HPLC |
|---|---|---|---|---|
| | | | | purity |
| Entry | Input | conditions | Temp./Time | (area %) |
| 1 | 42.0 mg | BCY8234: 0.1 g, 1.0 eq. | 17-23° C., 1.5 h | FP: 80.34% |
| | gvcMMAE | gvcMMAE: 42.0 mg, 1.0 eq. | 17-23° C., 16 h | FP: 78.97% |
| | (free acid) | TBTU: 10.9 mg, 1.0 eq. | | |
| | | DIEA: 8.7 + 39.4 mg, 2.0 + 9.0 eq. | | |
| | | DMA: 0.6 + 0.4 mL, 6.0 + 4.0 v | | |
| | Work up | DME: 5.6 mL, 56 v | 17-23° C., 3.0 h | FP: 80.36% |
| 2 | 45.4 mg | BCY8234: 0.1 g, 1.0 eq. | 17-23° C., 1.5 h | FP: 86.51% |
| | gvcMMAE | gvcMMAE: 45.4 mg, 1.0 eq. | 17-23° C., 16 h | FP: 81.88% |
| | (TEA salt) | TBTU: 10.9 mg, 1.0 eq. | | |
| | | DIEA: 8.7 + 39.4 mg, 2.0 + 9.0 eq. | | |
| | | DMA: 0.6 + 0.4 mL, 6.0 + 4.0 v | | |
| | Work up | DME: 5.6 mL, 56 v | 17-23° C., 3.0 h | FP: 86.25% |

TABLE 21

| | | HPLC/area % | | | | | | | | | | | | | | |
|---|---|---|---|---|---|---|---|---|---|---|---|---|---|---|---|---|
| | | | RRT | | | | | | | | | | | | | RRT |
| | | | 0.48 | RRT | RRT | RRT | RRT | RRT | RRT | RRT | | RRT | RRT | RRT | RRT | 1.23 |
| Entry | Conditions | Status | (BCY8234) | 0.63 | 0.70 | 0.88 | 0.93 | 0.97 | 0.97 | 0.99 | FP | 1.01 | 1.01 | 1.06 | 1.08 | (gvcMMAE) |
| | BT8009 | Solid | | | | | | 2.07 | | | 95.90 | 1.32 | | 0.58 | 0.12 | |
| | Reference | | | | | | | | | | | | | | | |
| | standard | | | | | | | | | | | | | | | |
| 1 | gvcMMAE | IPC @1.5 h | 1.93 | 1.07 | 0.17 | 4.81 | 0.29 | 0.55 | 0.66 | 0.49 | 80.34 | 1.63 | 1.82 | 0.33 | 1.23 | 0.11 |
| | free acid | IPC @16 h | 1.64 | 0.94 | 0.09 | 4.94 | 0.24 | 0.15 | 4.57 | — | 78.97 | 1.86 | 1.24 | 0.93 | 0.96 | — |
| | | Wet cake | 1.03 | 0.97 | 0.15 | 4.83 | 0.28 | 0.29 | 3.02 | — | 80.36 | 1.64 | 1.54 | 0.25 | 1.01 | — |
| 2 | gvcMMAE | IPC @1.5 h | 1.23 | 0.20 | — | 0.24 | 0.15 | 0.53 | 0.78 | 0.61 | 86.51 | 1.81 | 1.95 | 0.24 | 1.27 | 0.94 |
| | TEA salt | IPC @16 h | 1.30 | 0.16 | — | 0.50 | 0.11 | 0.15 | 3.20 | — | 81.88 | 1.79 | 1.44 | 2.05 | 1.33 | — |
| | | Wet cake | 0.41 | 0.16 | — | 0.47 | 0.18 | 0.33 | 2.95 | — | 86.25 | 2.09 | 1.96 | 0.28 | 1.11 | — |

3.3.13. Stress Test of gvcMMAE at Different Drying Temperature (Table 22 and 23)

Stress test of gvcMMAE at different drying temperatures was done. According to the result listed below, gvcMMAE was stable at 40° C. for 3 days.

TABLE 22

| | | | HPLC/area % | |
|---|---|---|---|---|
| Input | Conditions | Temp./Time | Status | gvcMMAE |
| Before | | | Dry | 98.54 |
| 0.05 g gvcMMAE | gvcMMAE: 0.05 g 20° C. | 20° C., 3 days | Dry | 98.62 |
| 0.05 g gvcMMAE | gvcMMAE: 0.05 g 30° C. | 30° C., 3 days | Dry | 98.65 |
| 0.05 g gvcMMAE | gvcMMAE: 0.05 g 40° C. | 40° C., 3 days | Dry | 98.61 |
| 0.05 ggvcMMAE | gvcMMAE: 0.05 g 50° C. | 50° C., 3 days | Dry | 98.52 |

TABLE 23

| | HPLC/area % | | | | | | | | | | | | |
|---|---|---|---|---|---|---|---|---|---|---|---|---|---|
| | | | | | | | | | | | | vcMMAE | |
| | | RRT | RRT | RRT | RRT | RRT | | RRT | RRT | RRT | RRT | RRT | RRT |
| Conditions | Status | 0.65 | 0.90 | 0.94 | 0.96 | 0.98 | gvcMMAE | 1.02 | 1.04 | 1.05 | 1.20 | 1.22 | 1.31 |
| Before | DRY | — | 0.05 | 0.04 | 0.13 | 0.31 | 98.54 | 0.15 | 0.13 | — | — | — | 0.58 |
| 20° C., 3 days | DRY | — | 0.06 | 0.04 | 0.10 | 0.32 | 98.62 | 0.16 | 0.14 | — | — | — | 0.50 |
| 30° C., 3 days | DRY | — | 0.07 | 0.04 | 0.11 | 0.32 | 98.65 | 0.12 | 0.14 | — | — | — | 0.51 |
| 40° C., 3 days | DRY | 0.02 | 0.08 | 0.04 | 0.12 | 0.30 | 98.61 | 0.13 | 0.14 | — | — | — | 0.50 |
| 50° C., 3 days | DRY | 0.11 | 0.08 | 0.03 | 0.11 | 0.32 | 98.52 | 0.14 | 0.14 | — | — | — | 0.50 |

### 3.3.14. 2^{nd} Round Process Optimization (Table 24 and 25)

A robust process was developed by using DMA/THF/TEA as the reaction system and THF/MTBE as the work up system.

One batch on a 5 g scale of vcMMAE was conducted to verify this process. The IPC purity was 98.37 A %. After work up, a good solid state was obtained and the purity of wet cake was 98.60 A %. After vacuum drying, 6.03 g (~0.2 g to do stress test, theory amount: 5.51 g) of gvcMMAE was obtained with 98.59 A % HPLC purity (Residual: TEA: 4.83%; THF: 0.01%; MTBE: 9.65%; DMA: 0.54%).

TABLE 24

| | | | | | HPLC | |
|---|---|---|---|---|---|---|
| Lot/ | | | | | purity | |
| Batch No. | Input | conditions | Temp./Time | | (area %) | Note |
| 1 & 2 | vcMMAE: 5.0 g | vcMMAE: 5.0 g, 1.0 eq. | −5~5° C., | 1 h | gvcMMAE: | |
| | | (assay corrected). | | | 98.39% | |
| | | Glutaric anhydride: | −5~5° C., | 16 h | gvcMMAE: | |
| | | 559 mg, 1.10 eq. | | | 98.37% | |
| | | TEA: 586 mg, 1.3 eq. | | | | |
| | | DMA: 1.25 mL, 0.25 v | | | | |
| | | THF: 18.75 mL, 3.75 v | | | | |
| | gvcMMAE | THF: 125 mL, 25 v | −5~5° C., | 1 h | gvcMMAE: | Good solid |
| | reaction | MTBE: 250 mL, 50 v | | | 98.60% | state after |
| | solution | | | | | stirring for 1 h. |
| | (5.0 g scale, | | | | | |
| | DMA:THF = | | | | | |
| | 0.25:3.75) | | | | | |

| | | | | |
|---|---|---|---|---|
| Vacuum drying | 23° C., | 16 h | gvcMMAE: | Dry cake: 6.4 g |
| | | | 98.54% | |

| | | |
|---|---|---|
| 30° C., | — | Dry cake: 6.1 g |
| another 16 h | | THF: 0.02% |
| | | MTBE: 10.5% |
| | | DMA: 0.69% |
| | | TEA: 4.37% |
| 30° C., | gvcMMAE: | Dry cake: 6.04 g |
| another 16 h | 98.59% | THF: 0.01% |
| | | MTBE: 9.65% |
| | | DMA: 0.54% |
| | | TEA: 4.83% |

TABLE 25

| | | HPLC/area % | | | | | | | | | | | | |
|---|---|---|---|---|---|---|---|---|---|---|---|---|---|---|
| Lot/ | | | | | | | | | | | RRT | | | |
| Batch | | | RRT | RRT | RRT | RRT | | RRT | RRT | RRT | 1.08 | RRT | RRT | RRT |
| No. | Conditions | Status | 0.90 | 0.94 | 0.96 | 0.98 | gvcMMAE | 1.02 | 1.04 | 1.05 | vcMMAE | 1.20 | 1.31 | 1.78 |
| 1 | Reaction | IPC1, 1 h | — | 0.05 | 0.13 | 0.34 | 98.39 | 0.17 | 0.15 | — | — | — | 0.59 | 0.09 |
| | DMA:THF = | IPC2, 16 h | 0.05 | 0.05 | 0.09 | 0.33 | 98.37 | 0.17 | 0.15 | — | — | — | 0.58 | 0.09 |
| | 0.25:3.75 | | | | | | | | | | | | | |
| 2 | Work up | Wet cake | 0.04 | 0.04 | 0.06 | 0.28 | 98.60 | 0.15 | 0.14 | — | — | — | 0.56 | — |
| | | | | | | 619.7 | 619.7 | | | | | | 782.0 | |
| | Vacuum | P,23° C., | 0.05 | 0.04 | 0.13 | 0.31 | 98.54 | 0.15 | 0.13 | — | — | — | 0.58 | — |
| | drying | 16 h | | | | | | | | | | | | |
| | | P3, 30° C., | 0.09 | 0.05 | 0.13 | 0.28 | 98.59 | 0.14 | 0.14 | — | — | — | 0.52 | — |
| | | another 32 h | | | | | | | | | | | | |

3.4. Scale Up with Optimized Process

### 3.4.1. Use Test of Raw Material (Table 26)

A use test of vcMMAE on a 1 g scale of vcMMAE was done. The IPC was normal: gvcMMAE: 98.61 A %. After work up, a good solid state was obtained, the purity of the wet cake was 98.45 A %. After vacuum drying for 16 h at 40° C., 1.12 g (Theory amount: 1.10 g) of gvcMMAE was obtained with 98.57 A % HPLC purity (Residual: TEA: 4.81%; THF: 0.07%; MTBE: 7.30%; DMA: 0.46%). After vacuum drying for another 16 h at 40° C., the purity of gvcMMAE was 98.47 A %. (Residual: TEA: 4.43%; THF: 0.05%; MTBE: 7.04%; DMA: 0.34%).

TABLE 26

| | | | | | |
|---|---|---|---|---|---|
| | | | | HPLC | |
| | | | | purity | |
| Entry | Input | conditions | Temp./Time | (area %) | Note |

| | | | | | |
|---|---|---|---|---|---|
| 1 | vcMMAE: 1.0 g | vcMMAE: 1.0 g, 1.0 eq. | −5~5° C., | 1 h | gvcMMAE: |
| | | (1.15 g, assay: 86.9%). | | | 98.61% |
| | | Glutaric anhydride: | | | |
| | | 112 mg, 1.10 eq. | | | |
| | | TEA: 118 mg, 1.3 eq. | | | |
| | | DMA: 0.25 mL, 0.25 v | | | |
| | | THF: 3.75 mL, 3.75 v | | | |
| | gvcMMAE | THF: 25 mL, 25 v | −5~5° C., | 1 h | gvcMMAE: |
| | reaction solution | MTBE: 50 mL, 50 v | | | 98.46% |
| | (1.0 g scale) | | | | |

| | | | | |
|---|---|---|---|---|
| Vacuum drying | 40° C., | 16 h | gvcMMAE: | Dry cake: 1.12 g |
| | | | 98.57% | THF: 0.07% |
| | | | | MTBE: 7.30% |
| | | | | DMA: 0.46% |
| | | | | TEA: 4.81% |

| | | |
|---|---|---|
| 40° C., | gvcMMAE: | THF: 0.05% |
| another 16 h | 98.47% | MTBE: 7.04% |
| | | DMA: 0.34% |
| | | TEA: 4.43% |

### 3.4.2. Scale Up on 1^{st} Batch (Table 27)

One scale-up batch was conducted on a 10 g scale of vcMMAE. The IPC was normal: gvcMMAE: 98.53 A %. After work up, 11.76 g of gvcMMAE was obtained with 98.51 A % HPLC purity in a 95.64% corrected yield. (Assay: 89.6%, Residual: TEA: 3.10%; THF: 0.93%; MTBE: 3.1%; DMA: 1.4%; glutaric anhydride: 0.06%; KF: 0.57%).

TABLE 27

| | | | | | |
|---|---|---|---|---|---|
| | | | | HPLC | |
| | | | | purity | |
| Entry | Input | conditions | Temp./Time | (area %) | Note |

| | | | | | |
|---|---|---|---|---|---|
| 1 | vcMMAE: 10.0 g | vcMMAE: 10.0 g, 1.0 eq. | −5~5° C., | 1 h | gvcMMAE: |
| | | (11.50 g, assay: 86.9%). | | | 98.53% |
| | | Glutaric anhydride: | | | |
| | | 1.12 g, 1.10 eq. | | | |
| | | TEA: 1.18 mg, 1.3 eq. | | | |
| | | DMA: 2.5 mL, 0.25 v | | | |
| | | THF: 37.5 mL, 3.75 v | | | |
| | gvcMMAE | THF: 250 mL, 25 v | −5~5° C., | 1 h | gvcMMAE: |
| | reaction solution | MTBE: 500 mL, 50 v | | | 98.67% |
| | (10.0 g scale) | | | | |

| | | | | |
|---|---|---|---|---|
| Vacuum drying | 40° C., | 16 h | gvcMMAE: | Dry cake: 12.00 g |
| | | | 98.53% | THF: 2.30% |
| | | | | MTBE: 3.95% |
| | | | | DMA: 1.39% |
| | | | | TEA: 2.02% |

| | | |
|---|---|---|
| 40° C., | gvcMMAE: | Dry cake: 11.76 g |
| another 16 h | 98.51% | (about 200 mg |
| | | for RS/IC test) |
| | | Assay: 89.6% |
| | | THF: 0.93% |
| | | MTBE: 3.1% |
| | | DMA: 1.4% |
| | | TEA: 3.10% |
| | | glutaric |
| | | anhydride: 0.06% |
| | | KF: 0.57% |

### 3.4.3. Scale Up on 2^{nd} Batch (Table 28 and 29)

One scale-up batch was conducted on a 30 g scale of vcMMAE. The IPC was normal: gvcMMAE: 98.51 A %. After work up, 36.53 g (~0.3 g for RS/IC test) of gvcMMAE was obtained with an 98.56 A % HPLC purity in 97.17% corrected yield. (Assay: 87.9%, Residual: TEA: 4.5%; THF: 1.8%; MTBE: 2.5%; DMA: 2.1%; glutaric anhydride: 0.09%; KF: 0.45%).

TABLE 28

| | | | | | |
|---|---|---|---|---|---|
| | | | | HPLC | |
| | | | | purity | |
| Entry | Input | conditions | Temp./Time | (area %) | Note |

| | | | | | |
|---|---|---|---|---|---|
| 1 | vcMMAE: 30.0 g | vcMMAE: 30.0 g, 1.0 eq. | −5~5° C., | 1 h | gvcMMAE: |
| | | (34.52 g, assay: 86.9%). | | | 98.51% |
| | | Glutaric anhydride: | | | |
| | | 3.36 g, 1.10 eq. | | | |
| | | TEA: 3.54 g, 1.3 eq. | | | |
| | | DMA: 7.5 mL, 0.25 v | | | |
| | | THF: 112.5 mL, 3.75 v | | | |
| | gvcMMAE | THF: 750 mL, 25 v | −5~5° C., | 1 h | gvcMMAE: |
| | reaction solution | MTBE: 1500 mL, 50 v | | | 98.50% |
| | (30.0 g scale) | | | | |

| | | | | |
|---|---|---|---|---|
| Vacuum drying | 40° C., | 16 h | gvcMMAE: | Dry cake: 37.59 g |
| | | | 98.46% | THF: 4.14% |
| | | | | MTBE: 2.71% |
| | | | | DMA: 1.74% |
| | | | | TEA: 2.62% |

| | | |
|---|---|---|
| 40° C., | gvcMMAE: | Dry cake: 36.53 g |
| another 16 h | 98.56% | (about 300 mg |
| | | for RS/IC test) |
| | | Assay: 87.9% |
| | | THF: 1.8% |
| | | MTBE: 2.5% |
| | | DMA: 2.1% |
| | | TEA: 4.5% |
| | | glutaric |
| | | anhydride: 0.09% |
| | | KF: 0.45% |

TABLE 29

| | HPLC/area % | | | | | | | | | | | | |
|---|---|---|---|---|---|---|---|---|---|---|---|---|---|
| | | | | | | | | | | | | vcMMAE | |
| | | RRT | RRT | RRT | RRT | RRT | RRT | RRT | RRT | RRT | RRT | RRT | RRT |
| Conditions | Status | 0.65 | 0.90 | 0.94 | 0.96 | 0.98 | 0.98 | 1.02 | 1.04 | 1.05 | 1.20 | 1.22 | 1.31 |
| 1 g scale | IPC | — | — | 0.02 | 0.15 | 0.22 | 98.61 | 0.15 | 0.14 | — | — | — | 0.60 |
| | WET | — | — | 0.03 | 0.14 | 0.24 | 98.46 | 0.20 | 0.17 | — | — | — | 0.64 |
| | DRY | — | 0.01 | 0.02 | 0.16 | 0.27 | 98.57 | 0.14 | 0.13 | — | — | — | 0.60 |
| | (40° C. 16 h) | | | | | | | | | | | | |
| | DRY2 | — | 0.06 | 0.03 | 0.14 | 0.33 | 98.46 | 0.15 | 0.12 | — | — | — | 0.63 |
| | (40° C. | | | | | | | | | | | | |
| | another16 h) | | | | | | | | | | | | |
| 10 g | IPC | — | — | 0.04 | 0.15 | 0.26 | 98.53 | 0.15 | 0.13 | — | — | — | 0.56 |
| scale up | WET | — | 0.01 | 0.03 | 0.12 | 0.21 | 98.67 | 0.16 | 0.14 | — | — | — | 0.60 |
| | DRY | — | 0.02 | 0.02 | 0.17 | 0.26 | 98.53 | 0.16 | 0.14 | — | — | — | 0.54 |
| | (40° C. 16 h) | | | | | | | | | | | | |
| | DRY2 | — | 0.03 | 0.03 | 0.17 | 0.26 | 98.51 | 0.16 | 0.14 | — | — | — | 0.54 |
| | (40° C. | | | | | | | | | | | | |
| | another16 h) | | | | | | | | | | | | |
| 30 g | IPC | — | — | 0.04 | 0.16 | 0.20 | 98.51 | 0.13 | 0.15 | — | — | — | 0.60 |
| scale up | WET | — | — | 0.04 | 0.16 | 0.25 | 98.50 | 0.16 | 0.14 | — | — | — | 0.58 |
| | DRY | — | 0.05 | 0.04 | 0.17 | 0.24 | 98.46 | 0.15 | 0.14 | — | — | — | 0.60 |
| | (40° C. 16 h) | | | | | | | | | | | | |
| | DRY2 | — | 0.05 | 0.03 | 0.13 | 0.30 | 98.56 | 0.17 | 0.14 | — | — | — | 0.49 |
| | (40° C. | | | | | | | | | | | | |
| | another16 h) | | | | | | | | | | | | |

### 3.5. Impurities Identification

LC-MS has been carried out to identify the impurities. The possible structure of impurity RRT 0.90 is shown below. The structure of other impurities are still unknown.

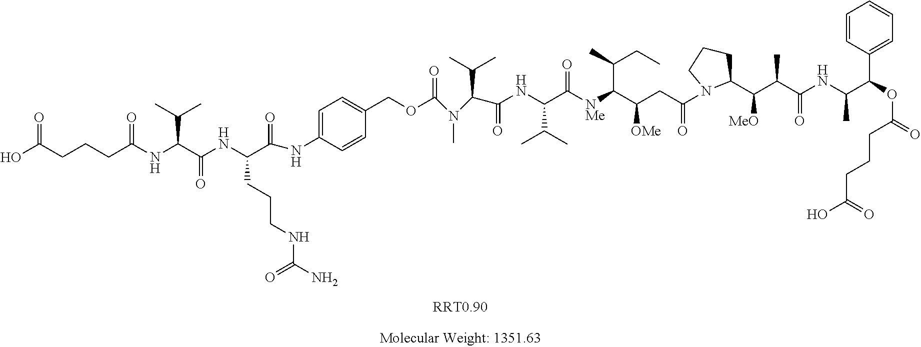

TABLE 30

| Lot/ | | HPLC/area % | | | | | | | | | | | | |
|---|---|---|---|---|---|---|---|---|---|---|---|---|---|---|
| Batch | | | RRT | RRT | RRT | RRT | RRT | RRT | RRT | RRT | RRT | RRT | vcMMAERRT | RRT |
| No. | Conditions | Status | 0.65 | 0.90 | 0.94 | 0.96 | 0.98 | 0.98 | 1.02 | 1.04 | 1.05 | 1.20 | 1.22 | 1.31 |
| Proposed | | | | MS: | | | MS: | J | | | | | | MS: |
| structure | | | | 677.1 | | | 619.7 | MS: | | | | | | 782.0 |
| | | | | | | | | 619.7 | | | | | | |

### 3.6. Typical Process

1. Charge DMAC/THF (120 mL, 0.25:3.75 v/v, 4.0 v) into R1 (reaction vessel 1).
2. Charge vcMMAE (30 g, assay corrected, 1.0 eq.) into R1.
3. Adjust R1 to −5~5° C.
4. Stir R1 for 0.1 h at −5~5° C.
5. Charge glutaric anhydride (3.36 g, 1.1 eq.) into R1.
6. Charge TEA (3.54 g, 1.3 eq.) into R1.
7. Stir R1 for 1 h at −5~5° C.
8. Take sample for analysis (IPC purity of gvcMMAE).
9. Charge THF (750 mL, 25 v) into R2 (reaction vessel 2).
10. Charge MTBE (1500 mL, 50 v) into R2.
11. Adjust R2 to −5~5° C.
12. Stir R2 for 0.1 h at −5~5° C.
13. Warm the R1 to 15~25° C. (The solution will become clear when warmed to the room temperature)
14. Transfer the solution from R1 to R2 dropwise over 1 h.
15. Stir R2 for 1 h at −5~5° C.
16. Filter and rinse the wet cake with MTBE (300 mL, 10 v).
17. Dry the wet cake at 40° C. for 16~32 h.
18. Take sample for release test.

### 3.6. Further Process Development

Reactions using 3.3 g of vcMMAE (1 eq.) were carried out with 1.5 eq. of glutaric anhydride. The impurity level in the gvcMMAE product corresponding of the compound of Formula (III) was quantified.

Increasing reaction temperature to 15-25° C. yielded IPC purity in reaction solution of 99.13 A % gvcMMAE (row A; table 31).

Crystallization with THF/MTBE (row B; table 31) led to a concentration of the compound of Formula (III) in the product gvcMMAE of 0.33 A % (wet), which increased to 0.71 A % following drying.

Experiments to rinse the dry cake with glutaric anhydride solution (0.1 eq. anhydride in THF/MTBE (1/2): 3 v) led to an increase in the level of the compound of Formula III, suggesting (without being bound by theory) that at least in part the impurity of Formula III may arise from the presence of excess glutaric anhydride. However, experiments to rinse out the glutaric anhydride using solvents MTBE (10 V), first MTBE/THF=2/1: 5 V then MTBE: 5 V; or first THF: 5 V then MTBE: 5 V also led to an increase in the level of the compound of Formula III.

Quenching the reaction with water (0.5 eq; 10 min; 15-25° C.) prior to crystallization led to a significant improvement, with the crystallised gvcMMAE product containing only 0.52 A % Formula (III) (Table 31, row C). Without being bound by theory, this may demonstrate that quenching the reaction with water prior to crystallization assists in preventing reaction with excess glutaric anhydride.

TABLE 31

| | | | | HPLC purity (area %) | | | |
|---|---|---|---|---|---|---|---|
| | | | | | | | Further |
| | Condition | Temp./Time | Stage | Formula III | gvcMMAE | vcMMAE | impurity |
| A | Reaction solution | 15~25° C./1 h | IPC | 0.07 | 99.13 | — | 0.57 |
| | vcMMAE: 3.3 g | | | | | | |
| | glutaric anhydride: 1.5 eq. | | | | | | |
| | TEA: 1.50 eq. | | | | | | |
| | DMA/THF: 0.25/3.75 V | | | | | | |
| B | Crystallization with | −5~5° C./0.5 h | Wet | 0.33 | 98.84 | — | 0.60 |
| | THF/MTBE = 1/2: 80 V | Dry at | dry | 0.71 | 98.33 | — | 0.60 |
| | | 25~35° C./16 h | | | | | |
| C | added H2O: 0.5 eq. | −5~5° C./0.5 h | Wet | 0.32 | 98.83 | — | 0.61 |
| | stirring for 10 min at | Dry at | dry | 0.52 | 98.43 | — | 0.64 |
| | 15~25° C.; | 25~35° C./16 h | | | | | |
| | Crystallization with | | | | | | |
| | THF/MTBE = 1/2: 80 V | | | | | | |

Further experiments were conducted using 1.4 g of vcMMAE (1 eq.) with 1.15 eq. of glutaric anhydride. The reaction was carried out using DMA/THF/MTBE (0.25/28.5/50 v) as reaction solvents. The impurity level in the gvcMMAE product corresponding of the compound of Formula (III) was quantified. The suspension state was good and the impurity level arising from the compound of Formula (III) did not increase with the extension of reaction time.

The impurity level corresponding to the further impurity in Table 31 was quantified. The impurity level was significantly further reduced to about 0.17-0.18% and did not increase with the extension of reaction time. See Table 32.

TABLE 32

| | | | | HPLC purity (area %) | | | |
|---|---|---|---|---|---|---|---|
| | | | | Formula | | | Further |
| | Condition | Temp./Time | Stage | III | gvcMMAE | vcMMAE | impurity |
| A | vcMMAE : 1.4 g | 15~25° C./1 h | IPC | — | 99.47 | 0.08 | 0.17 |
| | glutaric anhydride: 1.15 eq. | 15~25° C./9 h | IPC | — | 99.50 | 0.02 | 0.18 |
| | TEA: 1.30 eq. | | | | | | |
| | DMAc/THF/MTBE | | | | | | |
| | 0.25/28.5/50 V | | | | | | |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The following are numbered aspects of the invention:
1. A method of synthesizing Glutaryl-Val-Cit-PAB-MMAE (gvcMMAE):

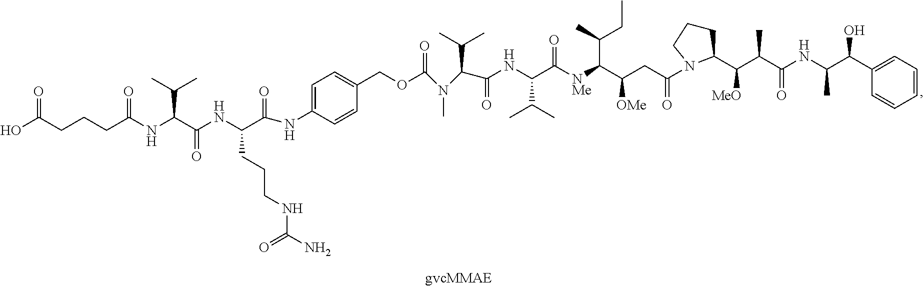

or a salt thereof, comprising adding glutaric anhydride into a solution comprising vcMMAE:

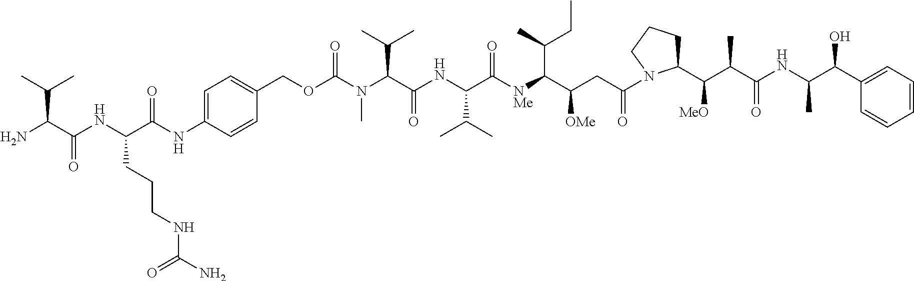

in a solvent comprising N,N-dimethylacetamide (DMA) and tetrahydrofuran (THF) to afford a reaction mixture.
2. The method of aspect 1, wherein the solvent is about 0.25:3.75 v/v DMA and THF (0.25:3.75 v/v DMA/THF).
3. The method of aspect 1 or 2, wherein glutaric anhydride is about 1.1 equivalent, and vcMMAE is about 1 equivalent.
4. The method of any one of aspects 1-3, further comprising adding about 1.3 equivalent TEA into the reaction mixture.
5. The method of any one of aspects 1-4, wherein the reaction between vcMMAE and glutaric anhydride is carried out at about −5° C. to about 5° C.
6. The method of aspect 5, wherein the reaction mixture is stirred for about 1 hour at about −5° C. to about 5° C. for the reaction to complete to afford a mixture comprising primarily gvcMMAE, or a gvcMMAE mixture.
7. The method of aspect 6, wherein the gvcMMAE mixture is warmed up to about 15° C. to about 25° C.
8. The method of aspect 6, wherein the gvcMMAE mixture is warmed up until it is a clear solution.
9. The method of aspect 7 or 8, wherein the gvcMMAE mixture is added into a mixture of THF and MTBE to afford gvcMMAE as a precipitate.
10. The method of aspect 9, wherein the mixture of THF and MTBE is at about −5° C. to about 5° C.
11. The method of aspect 9 or 10, wherein the mixture of THF and MTBE is about 1:2 v/v THF and MTBE (1:2 v/v THF/MTBE).
12. The method of aspect 11, wherein the volume ratio of the 0.25:3.75 v/v DMA/THF mixture to the 1:2 v/v THF/MTBE mixture is 4:75.
13. The method of any one of aspects 9-12, further comprising filtering out gvcMMAE precipitate as a wet filter cake, and rinsing the wet filter cake with MTBE.
14. The method of any one of aspects 1-13, further comprising reacting gvcMMAE with a Bicycle of Formula II:

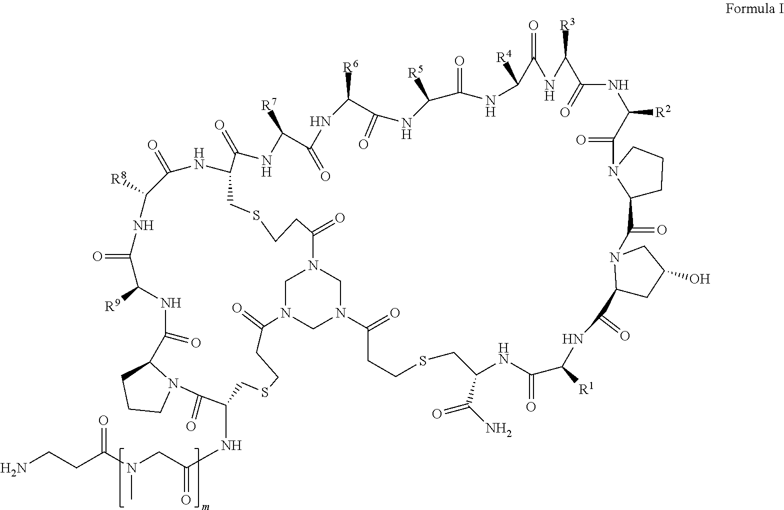

to form a Bicycle toxin conjugate of

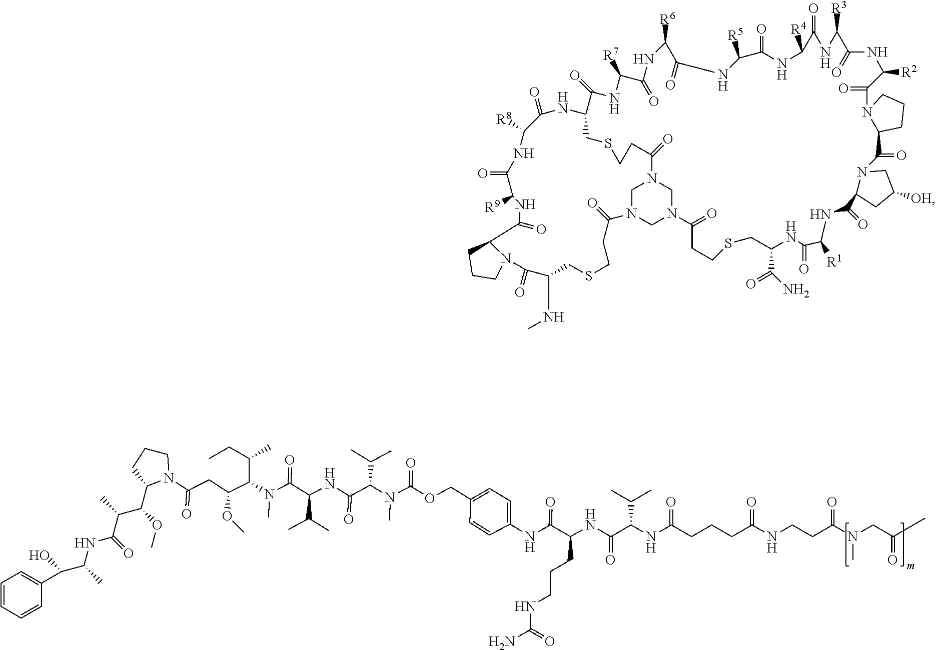

or a pharmaceutically acceptable salt thereof, wherein:
   each of R¹, R², R³, R⁴, R⁵, R⁶, R⁷, R⁸, and R⁹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
   m is 0, 1, 2, 3, 4, 5, 6, 7, 8, 9, 10, 11, 12, 13, 14, or 15.
15. The method of aspect 14, wherein the Bicycle toxin conjugate of Formula I is BT8009, or a pharmaceutically acceptable salt thereof.
16. The method of aspect 14, wherein the Bicycle toxin conjugate of Formula I is BT5528, or a pharmaceutically acceptable salt thereof.

We claim:

1. A method of synthesizing Glutaryl-Val-Cit-PAB-MMAE (gvcMMAE): 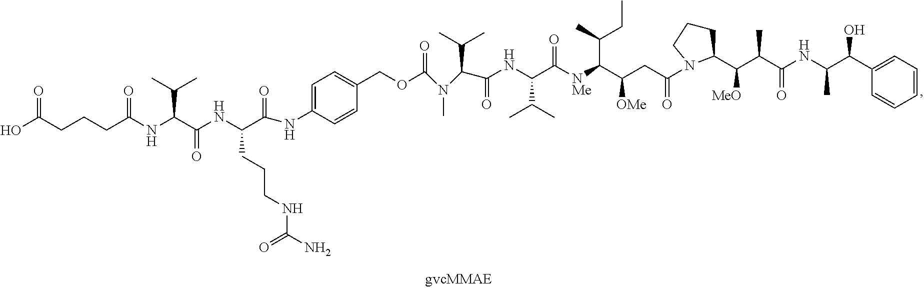
or a salt thereof, comprising adding glutaric anhydride into a solution comprising vcMMAE: 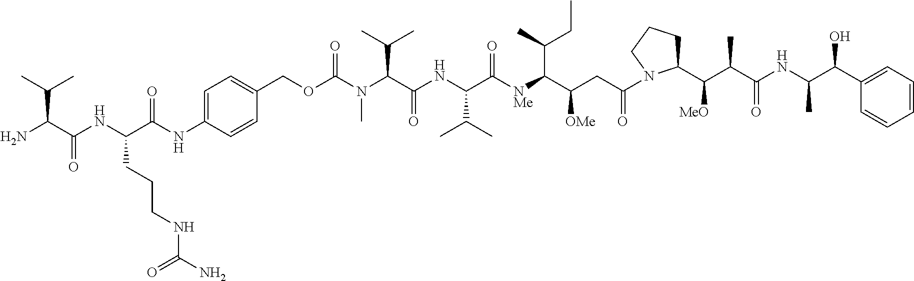
in a solvent comprising N,N-dimethylacetamide (DMA) and tetrahydrofuran (THF) to afford a reaction mixture.

2. The method of claim 1, wherein the solvent comprises THF and DMA; wherein the THF is present at about 3 to about 3.9 kg/kg (relative to the mass of vcMMAE) and the DMA is present at about 0.2 to about 0.3 kg/kg (relative to the mass of vcMMAE).

3. The method of claim 1 or 2, wherein the solvent comprises from about 0.2:3.9 to about 0.3-3 v/v DMA/THF,
optionally wherein the solvent is about 0.25:3.75 v/v DMA and THF (0.25:3.75 v/v DMA/THF).

4. The method of any one of the preceding claims, wherein the solvent further comprises methyl tert-butyl ether (MTBE)

5. The method of claim 1 or 4, wherein the solvent comprises DMA, THF and MTBE; wherein the DMA is present at about 0.2 to about 0.3 v; the THF is present at about 25 to about 35 v; and the DMA is present at about 30 to about 80 v, e.g. relative to the vcMMAE.

6. The method of any one of the preceding claims, comprising reacting vcMMAE with from about 1 to about 1.1 equivalents of glutaric anhydride;
optionally wherein glutaric anhydride is about 1.1 equivalent, and vcMMAE is about 1 equivalent.

7. The method of any one of the preceding claims, wherein the reaction mixture comprises from about 1.3 to about 1.5 equivalents of triethylamine (TEA);
optionally wherein the method comprises adding about 1.3 equivalent TEA into the reaction mixture.

8. The method of any one of the preceding claims, wherein the reaction between vcMMAE and glutaric anhydride is carried out at a temperature of from about −5° C. to about 25° C.

9. The method of any one of the preceding claims, where the reaction between vcMMAE and glutaric anhydride is carried out at a temperature of from about 15° C. to about 25° C.

10. The method of any one of the preceding claims, comprising stirring the reaction mixture during said reaction.

11. The method of any one of the preceding claims, wherein the reaction mixture is stirred for about 1 hour at about −5° C. to about 25° C. for the reaction to complete, optionally thereby affording a mixture comprising primarily gvcMMAE, or a gvcMMAE mixture.

12. The method of any one of the preceding claims, comprising quenching the reaction between vcMMAE and glutaric anhydride with water (H₂O).

13. The method of any one of the preceding claims, wherein quenching the reaction comprises adding from about 0.002 to about 0.01 kg/kg water (H₂O).

14. The method of claim 12 or 13, wherein the reaction between vcMMAE and glutaric anhydride is quenched with water at a temperature of from about 15 to about 25° C.

15. The method of any one of claims 12 to 14, wherein quenching the reaction comprises stirring the reaction mixture.

16. The method of any one of the preceding claims, wherein the gvcMMAE mixture is warmed up to about 15° C. to about 25° C.;
optionally wherein the gvcMMAE mixture is warmed up until it is a clear solution.

17. The method of any one of the preceding claims, wherein the gvcMMAE mixture is added into a mixture of THF and methyl tert-butyl ether (MTBE) to afford gvcMMAE as a precipitate.

18. The method of claim 17, wherein the mixture of THF and MTBE is at about −5° C. to about 5° C.

19. The method of claim 17 or 18, wherein the mixture of THF and MTBE comprises about 20 to about 25 kg/kg THF (relative to the mass of vcMMAE) and about 35 to about 39 kg/kg MTBE (relative to the mass of vcMMAE).

20. The method of any one of claims 17 to 19, wherein the mixture of THF and MTBE comprises from about 20:39 to about 25:30 v/v THF/MTBE;
optionally wherein the mixture of THF and MTBE is about 1:2 v/v THF and MTBE (1:2 v/v THF/MTBE).

21. The method of any one of claims 17 to 20, wherein the volume ratio of the DMA/THF mixture to the THF/MTBE mixture is from about 4:20 to about 4:200, e.g. about 4:75.

22. The method of any one of claims 17 to 21, further comprising filtering out gvcMMAE precipitate as a wet filter cake, and rinsing the wet filter cake with MTBE.

23. The method of any one of claims 1-22, further comprising reacting gvcMMAE with a Bicycle of Formula II: 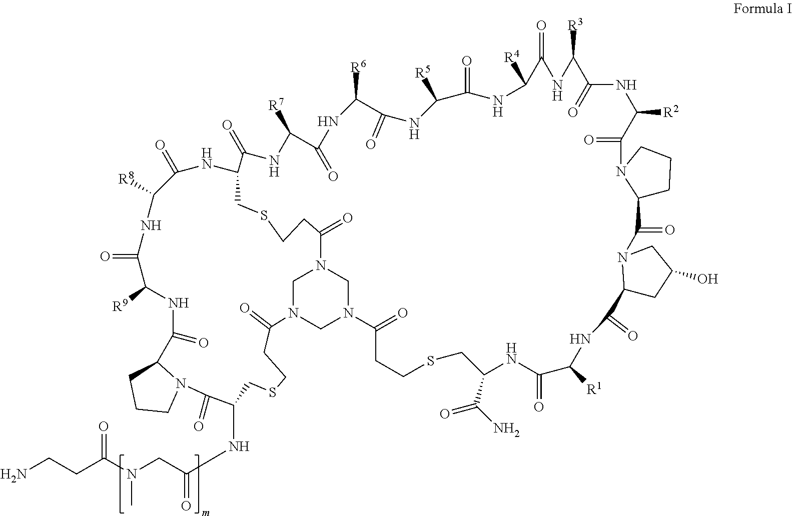
to form a Bicycle toxin conjugate of 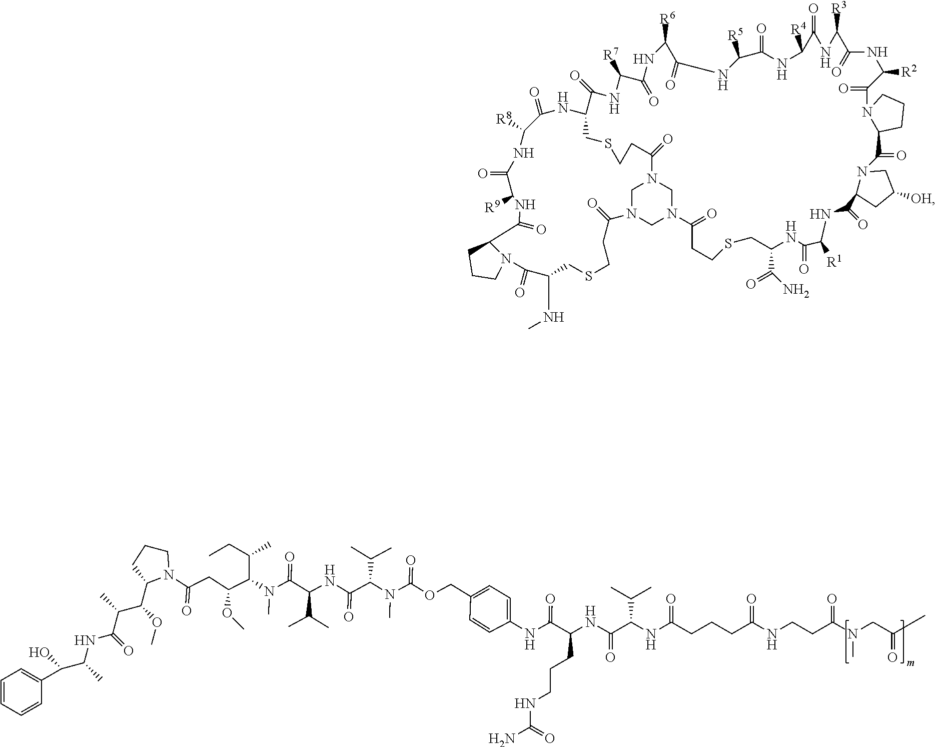
or a pharmaceutically acceptable salt thereof, wherein:
each of R¹, R², R³, R⁴, R⁵, R⁶, R⁷, R⁸, and R⁹ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
m is 0, 1, 2, 3, 4, 5, 6, 7, 8, 9, 10, 11, 12, 13, 14, or 15.

24. The method of claim 23, wherein the Bicycle toxin conjugate of Formula I is BT8009, or a pharmaceutically acceptable salt thereof.

25. The method of claim 23, wherein the Bicycle toxin conjugate of Formula I is BT5528, or a pharmaceutically acceptable salt thereof.

26. A compound, which compound is gvcMMAE, obtainable by the process of any one of claims 1 to 22.

27. A bicycle toxin conjugate of Formula (I), obtainable by the method of any one of claims 22 to 25.


SEQUENCE LISTING (4 sequences)

SEQ ID NO: 1 - protein, 26 aa, synthetic construct
  MOD_RES 1: note = bAla
  MOD_RES 2..11: note = Sarcosine
  MOD_RES 14: note = 1-napthylalanine
  SITE 15: note = D-Aspartic Acid
  MOD_RES 18: note = Homo-arginine
  MOD_RES 24: note = 4Hyp
  SITE 25: note = Amidated C-terminus
AGGGGGGGGGGCPADCMRDWSTPPWS

SEQ ID NO: 2 - protein, 29 aa, synthetic construct
  MOD_RES 1: note = bAla
  MOD_RES 2..11: note = Sarcosine
  MOD_RES 13: note = Homo-arginine
  MOD_RES 16: note = Hydroxyproline
  SITE 26: note = D-aspartic acid
  MOD_RES 28: note = Homo-arginine
AGGGGGGGGGGARDCPLVNPLCLHPDWRC

SEQ ID NO: 3 - protein, 15 aa, synthetic construct
  VARIANT 3..4: note = Side chain of X is independently hydrogen or an optionally substituted group selected from C1-6 aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.
  VARIANT 6..11: note = Side chain of X is independently hydrogen or an optionally substituted group selected from C1-6 aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.
  MOD_RES 13: note = 4Hyp
  VARIANT 14: note = Side chain of X is independently hydrogen or an optionally substituted group selected from C1-6 aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.
  SITE 15: note = Amidated C-terminus
CPXXCXXXXXXPPXC

SEQ ID NO: 4 - protein, 31 aa, synthetic construct
  MOD_RES 1: note = bAla
  MOD_RES 2..16: note = Sarcosine
  REGION 2..16: note = up to all of the sarcosine residues may be deleted
  VARIANT 19..20: note = Side chain of X is independently hydrogen or an optionally substituted group selected from C1-6 aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.
  VARIANT 22..27: note = Side chain of X is independently hydrogen or an optionally substituted group selected from C1-6 aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.
  MOD_RES 29: note = 4Hyp
  VARIANT 30: note = Side chain of X is independently hydrogen or an optionally substituted group selected from C1-6 aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.
  SITE 31: note = Amidated C-terminus
AGGGGGGGGGGGGGGGCPXXCXXXXXXPPXC